(19) United States
(12) Patent Application Publication
(10) Pub. No.: US 2023/0190761 A1
(43) Pub. Date: Jun. 22, 2023

(54) METHYLENE BLUE STABILIZED MRNA COMPOSITIONS

(71) Applicant: ModernaTX, Inc., Cambridge, MA (US)

(72) Inventors: Mark Brader, Cambridge, MA (US); Jessica Banks, Cambridge, MA (US)

(73) Assignee: ModernaTX, Inc., Cambridge, MA (US)

(21) Appl. No.: 17/926,353

(22) PCT Filed: May 21, 2021
(86) PCT No.: PCT/US2021/033644

§ 371 Date: Nov. 18, 2022

Related U.S. Application Data
(60) Provisional application No. 63/028,369, filed on May 21, 2020.

(51) Int. Cl. A61K 31/5415; A61K 31/7105; A61K 9/127; A61K 9/51; A61K 9/00; C12N 15/68

(52) U.S. Cl. CPC A61K 31/5415; A61K 31/7105; A61K 9/127; A61K 9/5123; A61K 9/0019; C12N 15/68

(57) ABSTRACT

Formulations of lipids and nucleic acids, including lipid nanoparticle formulations which encapsulate nucleic acids, stabilized with phenothiazinium dyes. In particular, the stabilization of LNP-mRNA formulations with methylene blue, Azure A, Azure B, Safranin O, phenosafranin or leucomethylene blue. Methods of making and of use of the formulations stabilized by chemical compounds are also provided.

### RELATED APPLICATIONS

This application claims the benefit under 35 U.S.C. § 119(e) of U.S. provisional application No. 63/028,369, filed May 21, 2020, which is incorporated by reference herein in its entirety.

### FIELD OF INVENTION

The present disclosures relate generally to formulations of lipids and nucleic acids, including lipid nanoparticle formulations which encapsulate nucleic acids, and more specifically to formulations stabilized by chemical compounds.

### BACKGROUND

The use of messenger RNA as a pharmaceutical agent is of great interest for a variety of applications, including in therapeutics, vaccines and diagnostics. Effective in vivo delivery of mRNA formulations represents a continuing challenge, as many such formulations are inherently unstable, activate an immune response, are susceptible to degradation by nucleases, or fail to reach their target organs or cells within the body due to issues with biodistribution. Each of these challenges results in loss of translational potency and therefore hinders efficacy of conventional mRNA pharmaceutical agents.

Various non-viral delivery systems, including nanoparticle formulations, present attractive opportunities to overcome many challenges associated with mRNA delivery. In particular, lipid nanoparticles (LNPs) have drawn particular attention in recent years as various LNP formulations have shown promise in a variety of pharmaceutical applications (Kowalski et al., “Delivering the Messenger: Advances in Technologies for Therapeutic mRNA Delivery” Molecular Therapy, 27(4):710-728 (2019); Gomez-Aguado, et al., “Nanomedicines to Deliver mRNA: State of the Art and Future Perspectives” Nanomaterials, 10, 264 (2020); Wadhwa et al., “Opportunities and Challenges in the Delivery of mRNA-Based Vaccines” Pharmaceutics, 12, 102 (2020)).

However lipids have been shown to degrade nucleic acids including mRNA, and lipid nanoparticle formulations undergo rapid loss of purity when stored as refrigerated liquids. It is also evident that the stability of mRNA is poorer when encapsulated within LNPs than when stored unencapsulated. It is generally regarded that a shelf life of at least 18 months is required for a viable pharmaceutical product, but mRNA formulations are not able to meet this stability mark, and consequently most mRNA formulations must be stored frozen at −20 C or −80 C, which is not ideal for patient-friendly or widespread use.

### SUMMARY OF THE INVENTION

The present invention provides, among other things, compositions and methods for the stabilization of nucleic acids. The invention encompasses, in some aspects, the observation that the mixture of various reactive compounds with lipid nanoparticle formulations comprising nucleic acids and/or nucleic acid formulations resulted in substantially improved formulation stability.

According to some aspects, stabilized pharmaceutical compositions are provided herein. In some embodiments, a stabilized pharmaceutical composition comprises a nucleic acid formulation comprising a nucleic acid and a lipid, and a compound of Formula I:

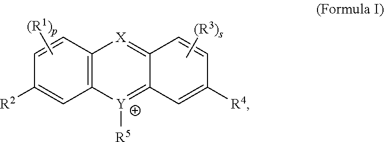

or an acceptable salt, tautomer, reduced form, or oxidized form thereof, mixed with the nucleic acid, wherein:

Y is N, S, or O;

X is N—R⁵, S, O, or C—R^{C};

R² and R⁴ are each independently —N(R^{N})₂;

each R⁵ is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or is absent;

each instance of R¹ and R³ is independently halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, optionally substituted sulfinyl, optionally substituted sulfonyl, —OR^{O}, —N(R^{N})₂, or —SR^{S};

p is 0, 1, 2, or 3;

s is 0, 1, 2, or 3;

each instance of R^{O} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or an oxygen protecting group;

each instance of R^{N} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or a nitrogen protecting group; or two R^{N} bonded to the name nitrogen atom are taken together with the intervening atoms to form optionally substituted heterocyclyl or optionally substituted heteroaryl;

each instance of R^{S} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or a nitrogen protecting group; and

R^{C} is hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, optionally substituted sulfinyl, optionally substituted sulfonyl, —OR^{O}, —N(R^{N})₂, or —SR^{S}.

In some embodiments, the nucleic acid formulation comprises lipid nanoparticles. In some embodiments, the nucleic acid formulation comprises liposomes. In some embodiments, the nucleic acid formulation comprises a lipoplex. In some embodiments, the nucleic acid is encapsulated within the lipid nanoparticles, liposomes, or lipoplex.

In some embodiments, the nucleic acid of the stabilized pharmaceutical composition is mRNA.

In some embodiments, the compound of a composition disclosed herein is a compound of Formula II:

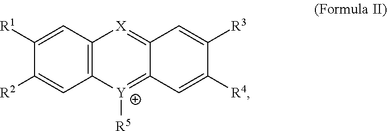

or an acceptable salt, tautomer, reduced form, or oxidized form thereof.

In some embodiments, the compound is a compound of Formula III:

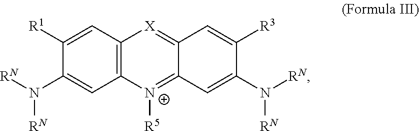

or an acceptable salt, tautomer, reduced form, or oxidized form thereof.

In some embodiments, the compound is not thionine or a salt thereof. In some embodiments, the compound is methylene blue, acriflavine, toluidine blue O, safranin O, phenosafranin or any mixture thereof.

In some embodiments, the compound is methylene blue, acriflavine, safranin O, phenosafranin or any mixture thereof.

In some embodiments, the compound is methylene blue.

In some embodiments, the compound has a purity of at least 70%, 80%, 90%, 95%, or 99%.

In some embodiments, the compound contains fewer than 100 ppm of elemental metals.

In some embodiments, a composition disclosed herein is formulated in an aqueous solution.

In some embodiments, the aqueous solution comprises lipid nanoparticles and the nucleic acid is encapsulated in the lipid nanoparticles.

In some embodiments, the aqueous solution has a pH of or about 5 to 8, including pH of about 5, 5.5, 6, 6.5, 7, 7.5, or 8.

In some embodiments, the aqueous solution does not comprise NaCl. In some embodiments, the aqueous solution comprises NaCl in a concentration of or about 150 mM.

In some embodiments, the aqueous solution comprises a phosphate buffer, a tris buffer, an acetate buffer, a histidine buffer, or a citrate buffer.

In some embodiments, the compound is present at a concentration between about 0.1 mM and about 3 mM in an aqueous solution. In some embodiments, the compound is present at a concentration of or about 2 mM in an aqueous solution. In some embodiments, the compound is present at a concentration of or about 1 mM in an aqueous solution. In some embodiments, the compound is present at a concentration of or about 0.5 mM in an aqueous solution.

In some embodiments, the nucleic acid of a composition disclosed herein is a lyophilized product.

In some embodiments, the lyophilized product comprises lipid nanoparticles and the nucleic acid is encapsulated in the lipid nanoparticles.

According to some aspects, stabilized pharmaceutical compositions are provided herein, comprising a nucleic acid formulation comprising a nucleic acid and a lipid, and methylene blue, having the formula:

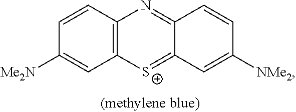 [SMILES: CN(C)c1ccc2nc3ccc(N(C)C)cc3[s+]c2c1]

mixed with the nucleic acid formulation.

In some embodiments, the nucleic acid formulation comprises lipid nanoparticles. In some embodiments, the nucleic acid formulation comprises liposomes. In some embodiments, the nucleic acid formulation comprises a lipoplex.

In some embodiments, the nucleic acid is encapsulated within the lipid nanoparticles, liposomes, or lipoplex. In some embodiments, the nucleic acid is mRNA.

In some embodiments, the methylene blue has a purity of at least 70%, 80%, 90%, 95%, or 99%. In some embodiments, the methylene blue contains fewer than 100 ppm of elemental metals.

In some embodiments, the composition is formulated in an aqueous solution. In some embodiments, the aqueous solution comprises lipid nanoparticles and the nucleic acid is encapsulated in the lipid nanoparticles. In some embodiments, the aqueous solution has a pH of or about 5 to 8, including pH of about 5, 5.5, 6, 6.5, 7, 7.5, or 8. In some embodiments, the aqueous solution does not comprise NaCl. In some embodiments, the aqueous solution comprises NaCl in a concentration of or about 150 mM. In some embodiments, the aqueous solution comprises a phosphate buffer, a tris buffer, an acetate buffer, a histidine buffer, or a citrate buffer.

In some embodiments, the methylene blue is present at a concentration between about 0.1 mM and about 3 mM. In some embodiments, the methylene blue is present at a concentration of or about 2 mM. In some embodiments, the methylene blue is present at a concentration of or about 1 mM. In some embodiments, the methylene blue is present at a concentration of or about 0.5 mM.

In some embodiments, the nucleic acid is a lyophilized product. In some embodiments, the lyophilized product comprises lipid nanoparticles and the nucleic acid is encapsulated in the lipid nanoparticles.

According to some aspects, compositions disclosed herein are used for the treatment of a disease in a subject. In some embodiments, the disease is caused by an infectious agent. In some embodiments, the disease is caused by or associated with a virus. In some embodiments, the disease is a disease caused by or associated with a malignant cell. In some embodiments, the disease is cancer.

According to some aspects, compositions having properties which inhibit microbial growth are disclosed herein. In some embodiments, microbial growth in a composition disclosed herein is inhibited by a compound disclosed herein. In some embodiments, a composition disclosed herein does not comprise phenol, m-cresol, or benzyl alcohol.

According to some aspects, methods of formulating nucleic acids are disclosed herein. In some embodiments, a method of formulating a nucleic acid comprises adding to a composition comprising a nucleic acid and a lipid, a compound of Formula I:

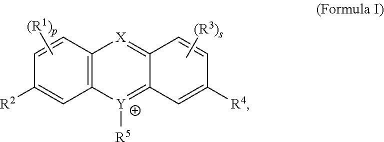

or an acceptable salt, tautomer, reduced form, or oxidized form thereof, wherein:

Y is N, S, or O;

X is N—R⁵, S, O, or C—R^{C};

R² and R⁴ are each independently —N(R^{N})₂;

each R⁵ is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or is absent;

each instance of R¹ and R³ is independently halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, optionally substituted sulfinyl, optionally substituted sulfonyl, —OR^{O}, —N(R^{N})₂, or —SR^{S};

p is 0, 1, 2, or 3;

s is 0, 1, 2, or 3;

each instance of R^{O} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or an oxygen protecting group;

each instance of R^{N} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or a nitrogen protecting group; or two R^{N} bonded to the name nitrogen atom are taken together with the intervening atoms to form optionally substituted heterocyclyl or optionally substituted heteroaryl;

each instance of R^{S} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or a nitrogen protecting group; and

R^{C} is hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, optionally substituted sulfinyl, optionally substituted sulfonyl, —OR^{O}, —N(R^{N})₂, or —SR^{S}, to prepare a formulated composition comprising the nucleic acid and the lipid.

In some embodiments, the formulated composition comprises lipid nanoparticles. In some embodiments, the formulated composition further comprises liposomes. In some embodiments, the formulated composition further comprises a lipoplex. In some embodiments, the nucleic acid is encapsulated in the lipid nanoparticles, liposomes, or lipoplex.

In some embodiments, the method further comprises subsequently removing the compound of Formula I from the formulated composition.

In some embodiments, the compound is a compound of Formula II:

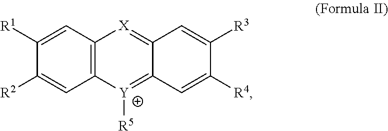

or an acceptable salt, tautomer, reduced form, or oxidized form thereof.

In some embodiments, the compound is a compound of Formula III:

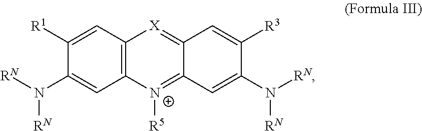

or an acceptable salt, tautomer, reduced form, or oxidized form thereof.

In some embodiments, the compound is not thionine or a salt thereof. In some embodiments, the compound is methylene blue, acriflavine, toluidine blue O, safranin O, phenosafranin, leucomethylene blue, or any mixture thereof. In some embodiments, the compound is methylene blue, acriflavine, safranin O, phenosafranin, leucomethylene blue, or any mixture thereof. In some embodiments, the compound is methylene blue.

In some embodiments, the compound has a purity of at least 70%, 80%, 90%, 95%, or 99%.

In some embodiments, the compound contains fewer than 100 ppm of elemental metals.

In some embodiments, the composition is formulated in an aqueous solution. In some embodiments, the aqueous solution comprises lipid nanoparticles and a nucleic acid is encapsulated in the lipid nanoparticles. In some embodiments, the aqueous solution has a pH of or about 5 to 8, including pH of about 5, 5.5, 6, 6.5, 7, 7.5, or 8. In some embodiments, the aqueous solution does not comprise NaCl. In some embodiments, the aqueous solution comprises NaCl in a concentration of or about 150 mM. In some embodiments, the aqueous solution comprises a phosphate buffer, a tris buffer, an acetate buffer, a histidine buffer, or a citrate buffer.

In some embodiments, the compound is present at a concentration between about 0.1 mM and about 3 mM in an aqueous solution. In some embodiments, the compound is present at a concentration of or about 2 mM in an aqueous solution. In some embodiments, the compound is present at a concentration of or about 1 mM in an aqueous solution. In some embodiments, the compound is present at a concentration of or about 0.5 mM in an aqueous solution.

In some embodiments, the composition is a lyophilized product. In some embodiments, the lyophilized product comprises lipid nanoparticles. In some embodiments, the lipid nanoparticles encapsulate a nucleic acid.

According to some aspects, methods of processing mRNA-lipid nanoparticles are provided herein. In some embodiments, pharmaceutically acceptable methods of processing an mRNA-lipid nanoparticle for therapeutic injection are provided, comprising adding a reactive compound to a lipid nanoparticle, and subsequently adding an mRNA to the lipid nanoparticle-reactive compound mixture, wherein the reactive compound sequesters degradative species of the lipid nanoparticle. In some embodiments, pharmaceutically acceptable methods of conferring anti-microbial properties to an mRNA-lipid nanoparticle composition are provided, comprising adding a reactive compound to the mRNA-lipid nanoparticle composition. In some embodiments, a pharmaceutically acceptable method of processing an mRNA-lipid nanoparticle for therapeutic injection comprises adding an mRNA to a lipid nanoparticle, and subsequently adding a reactive compound to the lipid nanoparticle-mRNA mixture, wherein the reactive compound sequesters degradative species of the lipid nanoparticle. In some embodiments, a pharmaceutically acceptable method of processing an mRNA-lipid nanoparticle for therapeutic injection comprises combining an mRNA, a lipid nanoparticle, and a reactive compound, wherein the reactive compound sequesters degradative species of the lipid nanoparticle.

According to some aspects compositions of lipid nanoparticles and mRNA having certain mRNA purity levels are provided herein. In some embodiments, a composition comprises a lipid nanoparticle encapsulating a mRNA, wherein the composition comprises a mRNA purity level of greater than 50% main peak mRNA purity after at least thirty days of storage.

In some embodiments, the composition comprises a mRNA purity level of greater than 60% main peak mRNA purity after at least thirty days of storage. In some embodiments, the composition comprises a mRNA purity level of greater than 70% main peak mRNA purity after at least thirty days of storage. In some embodiments, the composition comprises a mRNA purity level of greater than 80% main peak mRNA purity after at least thirty days of storage. In some embodiments, the composition comprises a mRNA purity level of greater than 90% main peak mRNA purity after at least thirty days of storage. In some embodiments, the composition comprises a mRNA purity level of greater than 50% main peak mRNA purity after at least six months of storage.

In some embodiments, the storage is at room temperature. In some embodiments, the storage is at greater than room temperature. In some embodiments, the storage is at 4° C.

In some embodiments, the composition comprises a phenothiazinium dye. In some embodiments, the phenothiazinium dye is not thionine or a salt thereof. In some embodiments, the phenothiazinium dye is methylene blue.

According to some aspects, compositions of lipid nanoparticles encapsulating mRNA having certain compositions of RNA fragments are provided herein. In some embodiments, a composition comprises a lipid nanoparticle encapsulating a mRNA, wherein the composition comprises less than 50% RNA fragments after at least thirty days of storage. In some embodiments, the composition comprises less than 60% RNA fragments after at least thirty days of storage. In some embodiments, the composition comprises less than 70% RNA fragments after at least thirty days of storage. In some embodiments, the composition comprises less than 80% RNA fragments after at least thirty days of storage. In some embodiments, the composition comprises less than 90% RNA fragments after at least thirty days of storage. In some embodiments, the composition comprises less than 95% RNA fragments after at least thirty days of storage.

In some embodiments, the composition is stored for at least six months.

In some embodiments, the storage is at room temperature. In some embodiments, the storage is at greater than room temperature. In some embodiments, the storage is at 4° C.

In some embodiments, the composition comprises a phenothiazinium dye. In some embodiments, the phenothiazinium dye is not thionine or a salt thereof. In some embodiments, the phenothiazinium dye is methylene blue.

In some embodiments, the lipid nanoparticle comprises a ratio of 20-60% amino lipids, 5-30% phospholipid, 10-55% structural lipid, and 0.5-15% PEG-modified lipid. In some embodiments, the lipid nanoparticle comprises a ratio of 20-60% amino lipids, 5-25% phospholipid, 25-55% structural lipid, and 0.5-15% PEG-modified lipid.

According to some aspects, methods for producing a protein in a subject are provided herein. In some embodiments, a method for producing a protein in a subject comprises administering a composition comprising a nucleic acid as disclosed herein to a subject, wherein the nucleic acid is an mRNA and wherein the mRNA encodes for the production of a protein in the subject.

According to some aspects, devices enabling the use of compositions and methods disclosed herein are provided. In some embodiments, a syringe or cartridge, comprising a composition disclosed herein is provided. In some embodiments, an infusion pump, comprising a composition disclosed herein is provided. In some embodiments, a syringe or cartridge, comprising multiple doses of a composition disclosed herein is provided.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B show mRNA instability in lipid nanoparticle formulations at refrigerated temperature. In each case less than 9 months of refrigerated storage stability would be possible. FIG. 1A shows sized-based purity of formulation 1 over 12 months of refrigerated storage. FIG. 1B shows sized-based purity of formulation 2 over 6 months of refrigerated storage.

FIG. 2 shows the chemical structure of methylene blue and thionine.

FIG. 3 shows a comparison of the stabilities of a series of mRNA LNP samples containing incremental quantities of thionine or methylene blue dyes exposed to two stress conditions (RT=room temperature, and 40° C.) for 3 weeks. Data are fragmentation analysis showing % main peak purity. The results show that thionine accelerated the degradation of mRNA in the LNP while methylene blue stabilized it, both at room temperature and 40° C.

FIG. 4 shows DLS characterization of mRNA-LNP samples after storage at different temperature conditions for 10 days in the presence of varying concentrations of methylene blue. These results show that the presence of up to 6 mM methylene blue had no significant effect on hydrodynamic size of the LNP as measured by DLS, and all samples remained highly monodisperse. These data support the conclusion that the presence of up to 6 mM methylene blue does not impact the physical stability of the LNP under the tested temperature conditions (5° C., 25° C., and 40° C.).

FIG. 5 shows results of fragment analysis on the sample set tested in FIG. 4, as described in Table 2. These data correspond to 10 days exposure to each temperature condition, followed by storage at 5° C. for 3 days prior to analysis.

FIG. 6 shows tabulated results of quantification of mRNA fragment analysis of mRNA-LNP samples incubated with varying concentrations of methylene blue at room temperature (RT), 5° C. or 40° C. These data correspond to 10 days exposure to each temperature condition, followed by storage at 5° C. for 3 days prior to analysis.

FIGS. 7A-7B shows results of LC/MS analysis of PEG-lipid incubated with varying concentrations of methylene blue for 1 week at room temperature (FIG. 7A) and 5° C. (FIG. 7B). The data represent LC/MS signal counts for PEG-lipid (half-filled circles) or Lyso-PEG-DMG degradation product (filled squares), normalized to total signal counts (PEG-lipid+Lyso-PEG-DMG). These data show that incremental concentrations of methylene blue (0, 0.1, 0.5 and 1 mM) had no effect on the total quantity of PEG-lipid present at either temperature condition. The data also show that the presence of methylene blue has no impact on formation of Lyso PEG-DMG. The data demonstrate that there is no significant adverse impact of the presence of methylene blue up to at least 1 mM on PEG-lipid stability.

FIG. 8 shows DLS intensity profiles of 5 mRNA-LNP samples after 3 weeks refrigerated storage. These data that the presence of dye in the range of 0-2 mM does not impact physical stability of the lipid nanoparticles.

FIG. 9 shows average DLS results for samples after 3 weeks of refrigerated storage. Mean and standard deviation values are shown for 3 independent measurements. These results show that the LNP is physically stable in the presence of dye and there was no change relative to the sample without dye.

FIGS. 10A-10B show results of two types of purity analysis applied to mRNA-LNP samples. FIG. 10A shows main peak purity analysis (sum of the two component peaks) by reverse phase (RP) chromatography. FIG. 10B shows results of fragment analysis on mRNA-LNP samples stored at 40° C. or −80° C. for 12 days. These results show that all samples stored at −80° C. exhibited equivalent levels of purity indicating that the presence of methylene blue does not interfere with analysis. Samples stored protected versus unprotected from light (“40 light”) show significantly different stability profiles. Samples stored protected from light (“40 dark”) show that the presence of methylene blue stabilized mRNA significantly with respect to loss of purity. Both methods show consistent trends and it can be inferred from the RP results that the presence of methylene blue does not have an adverse impact on the formation of RNA-lipid adducts. The light exposed condition utilized in this study corresponds to clear glass vials placed directly on the shelf in a 40° C. oven in front of its glass door.

FIGS. 11A-11B show impurity profiles of various mRNA-LNP samples stored at −80° C. (“Minus 80”), 40° C. protected from light (“40 dark”), or 40° C. exposed to light (“40 light”). The percent contributions of the individual components of the impurity profile are shown, as analyzed by RP-HPLC (FIG. 11A) or Fragment Analyzer (FIG. 11B).

FIG. 12 shows results of DLS measurements on samples after being stored for 5 months refrigerated (A-E) compared to a vial measured after being freshly thawed from storage at −80° C. DLS measurements were recorded in PBS after a 0.8 μm filtration to remove large particulates.

FIGS. 13A-13C show total purity as determined by main peak percentage (FIG. 13A), mRNA fragmentation (FIG. 13B) and mRNA adduct formation (FIG. 13C) in mRNA-LNP compositions with varying concentrations of methylene blue after 5 months (151 days) of refrigerated storage, as analyzed by RP-HPLC. The data demonstrate that concentrations of methylene blue as low as 0.5 mM significantly inhibit mRNA fragmentation rate relative to LNP compositions lacking dye. Further, there is little difference in the effect across the range of 0.5 to 2.0 mM methylene blue. The data suggest that a concentration of 1.5 mM is optimal with respect to inhibiting adduct formation.

FIG. 14 shows RNA integrity in lyophilized powder mRNA-LNP compositions with and without methylene blue at various storage temperatures (−80° C., 5° C. and 40° C.). Integrity was determined according to fragmentation, main peak percentage, and adduct formation, as measured by RP-HPLC. The data demonstrate that compositions containing methylene blue had improved mRNA purity (30% versus 22% main peak after 1 week at 40° C.).

FIG. 15 shows mRNA-LNP lyophilized powder compositions containing incremental concentrations of methylene blue.

FIG. 16 shows main peak purity of mRNA-LNP samples stored at 40° C. for ˜1 week in various buffer, pH and salt conditions, as determined by RP-HPLC.

FIG. 17 shows the effect of conventional antioxidants methionine (5 mM) and potassium metabisulfite (KDS, 5 mM) in the presence or absence of methylene blue (2 mM) on stabilization of mRNA-LNP formulations.

FIG. 18 shows differential scanning calorimetry thermograms of mRNA in the presence of no methylene blue, 50 μM methylene blue, or 100 μM methylene blue.

FIG. 19 shows circular dichroism spectra of mRNA in the presence of no methylene blue, 210 μM methylene blue, or 367 μM methylene blue. The spectra show various changes depending on the amount of methylene blue added, suggesting complexity in the interaction and more than a single type of binding.

FIG. 20 shows differential scanning calorimetry thermograms of 0.3 μM mRNA in the presence of 32.5 mM sodium acetate, pH 5.0, with various concentrations of methylene blue. The thermograms demonstrate the effect of incremental amounts of methylene blue on the mRNA. At 367.8 μM and above, there is a dramatic global change in the overall mRNA structure.

FIG. 21 shows cryo-electron micrographs of mRNA-LNPs with or without thionine.

FIG. 22 shows cryo-electron micrographs of mRNA-LNPs with or without thionine, demonstrating compaction of mRNA inside mRNA-LNPs in the presence of thionine.

FIG. 23 shows differential scanning calorimetry thermograms of 0.3 μM mRNA with and without 100 μM methylene blue. The thermograms show the effect of methylene blue on the structure of the mRNA is reversible by dialysis of the mRNA to remove methylene blue. The arrow indicates the normalization of the mRNA after MB dialysis curve to the peak of the mRNA only curve.

### DETAILED DESCRIPTION

Lipid nanoparticle (LNP) formulations offer the opportunity to deliver various nucleic acids in vivo for applications in which unencapsulated nucleic acids would be ineffective, but their broad utility has been hindered by insufficient nucleic acid stability over relevant timeframes. Degradation of nucleic acids within LNP formulations limits the use of such formulations to applications in which frozen compositions are acceptable. Whether LNP formulations could be amenable to long-term storage in refrigerated conditions remains unclear.

The present disclosure is based, at least in part, on the surprising finding that the mixture of various reactive compounds with nucleic acids in LNP formulations or with LNP formulations resulted in substantially improved stability including nucleic acid stability. Accordingly, provided herein are nucleic acid and lipid compositions and methods for their preparation and use.

It has been shown herein, using both accelerated and real-time conditions, that the stability of formulations can be significantly enhanced using the potent stabilizing excipients or compounds described herein. The inclusion of these compounds in formulations such as lipid based and/or nucleic acid formulations provides properties useful for preparation, storage and use of therapeutic agents. For instance, it has been demonstrated that for mRNA-lipid nanoparticle (mRNA-LNP) compositions, combination with the stabilizing compounds of the invention dramatically inhibit the rate of purity loss of mRNA encapsulated within the LNP under a variety of storage conditions. The instability of mRNA, specifically loss of purity, is considered one of the greatest challenges to its fundamental therapeutic and commercial viability. Additionally, the instability of mRNA is significantly worsened when formulated as an LNP. The stabilizing compounds of the invention provide a significant solution to these problems.

The discovery that a class of compounds is able to stabilize nucleic acids within a lipid carrier such as an LNP is thus unexpected and unprecedented. This finding enables several significant applications, including extended refrigerated liquid shelf-life, extended in-use periods at room temperature, and extended in-use stability at physiological temperatures up to higher temperatures such as 40° C. Achieving a stable liquid formulation also enables commercially and therapeutically desirable packaging and delivery options including prefilled syringes and cartridges for patient-friendly autoinjector and infusion pump devices. The incorporation of members of this class of compounds into methods of making as well as optionally, the final drug product will provide a significant improvement in purity values of therapeutic nucleic acids, such as mRNA upon manufacture. This solves a critical problem, as current manufacturing processes and formulations experience a 5-10% purity loss during LNP formation and processing that is typical with current large-scale LNP production. The ability to stabilize solutions and pharmaceutical preparations of mRNA and other therapeutics therefore represent a valuable technology facilitating broader use of therapeutic compositions such as mRNA compositions.

Some aspects of the present disclosure provide stabilized nucleic acid compositions comprising a nucleic acid and a compound of Formula I:

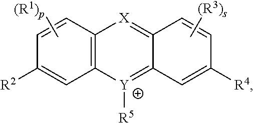

or an acceptable salt or tautomer thereof. R¹, R², R³, R⁴, R⁵, X, Y, p, and s are as described above. In certain embodiments, when X is N—R⁵, then at least one instance of R⁵ is absent.

The term “tautomers” or “tautomeric” refer to isomers of a compound which differ only in the position of the protons and electrons, e.g., two or more interconvertible compounds resulting from at least one migration of a hydrogen atom or electron pair, and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may be catalyzed by acid or base. Exemplary tautomerizations include keto-to-enol, amide-to-imide, lactam-to-lactam, enamine-to-imine, and enamine-to-(a different enamine) tautomerizations. Tautomerizations may result from delocalization of electrons (e.g., between heteroatoms and/or pi bonds in conjugated systems). According to the current disclosure, a non-limiting example of a tautomer of Formula I includes the formula:

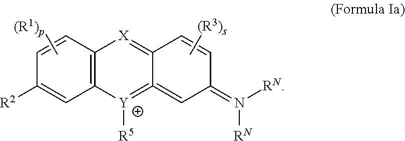

R¹, R², R³, R⁵, R^{N}, X, Y, p, and s are as described above, and R^{N} is optionally any structure described according to R¹, R², R³, R⁴ or R⁵ as described above. Chemical structures are further described below.

The term “reduced form” when used herein with respect to a compound refers to a derivative of said compound resulting from a decrease in oxidation state of one or more atoms of said compound, e.g., due to loss of electrons from said compound. According to the current disclosure, a non-limiting example of a reduced form of Formula I includes the formula:

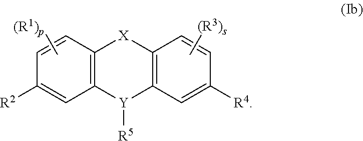

The term “oxidized form” when used herein with respect to a compound refers to a derivative of said compound resulting from an increase in oxidation state of one or more atoms of said compound, e.g., due to the compound gaining electrons. According to the current disclosure, a non-limiting example of an oxidized form of Formula Ib is Formula I. For further illustrative purposes, methylene blue is considered an “oxidized form” of leucomethylene blue, whereas leucomethylene blue is considered a “reduced form” of methylene blue:

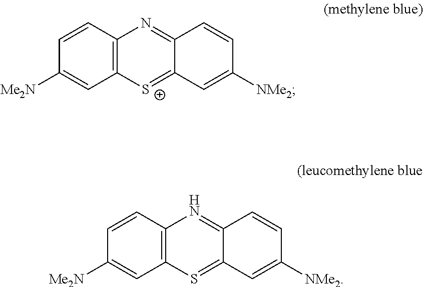

According to some aspects of the present disclosure, the compound is a compound of Formula II:

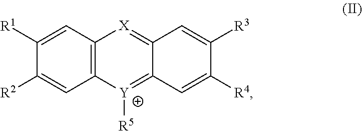

or an acceptable salt, tautomer, reduced form, or oxidized form thereof. In some embodiments, the compound is a compound of Formula IIb:

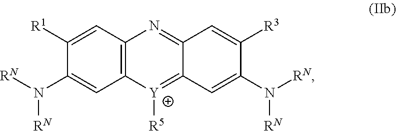

or an acceptable salt, tautomer, reduced form, or oxidized form thereof. In some embodiments, the compound is a compound of Formula III:

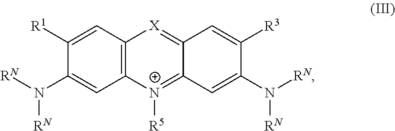

or an acceptable salt, tautomer, reduced form, or oxidized form thereof. R¹, R², R³, R⁴, R⁵, R^{N}, X and Y are as described above. In some embodiments, the compound is methylene blue, thionine acetate, Azure A chloride, Azure B, Toluidine Blue O, Safranin O, New methylene blue N, Acridine Orange hydrochloride hydrate, Proflavine hemisulfate salt hydrate, Acriflavine hydrochloride, 1,9-Dimethyl-Methylene Blue zinc chloride double salt, Nile Blue A, Nile Red, Bromophenol Blue sodium salt, Brilliant Blue G, Hematoxylin, Neutral Red, Crystal Violet, Phenol Red, Eosin B, Carmine, Fluorescein sodium salt, Methylene green zinc chloride double salt, Pyronin Y, or Leucomethylene Blue (mesylate). In some embodiments, the compound is methylene blue, acriflavine, toluidine blue O, safranin O, phenosafranin, or any combination or mixture thereof. In some embodiments, the compound is

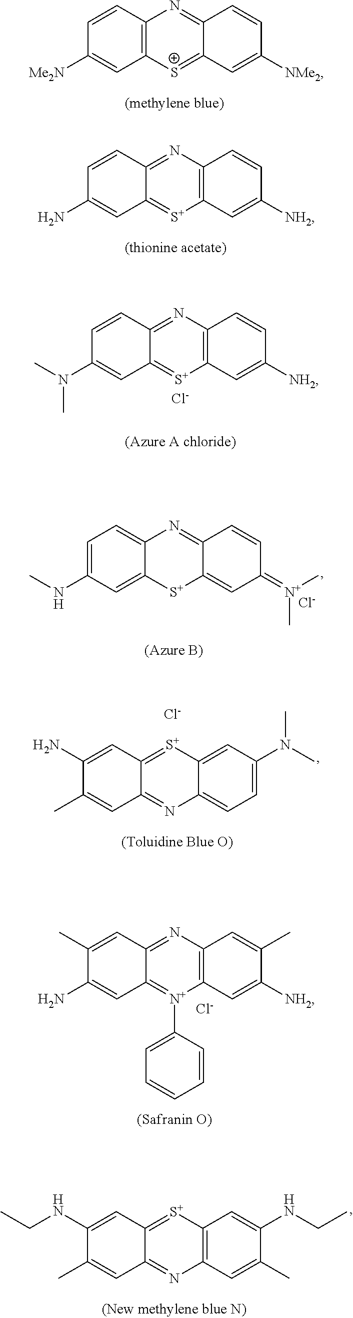

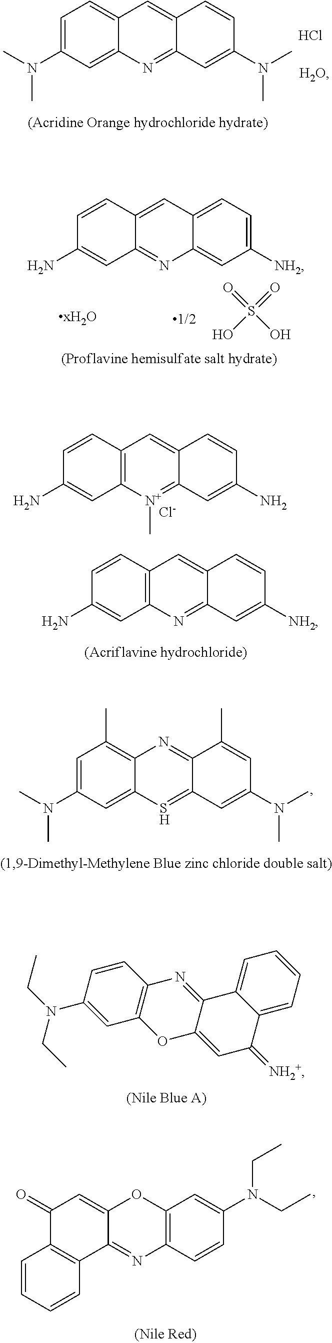

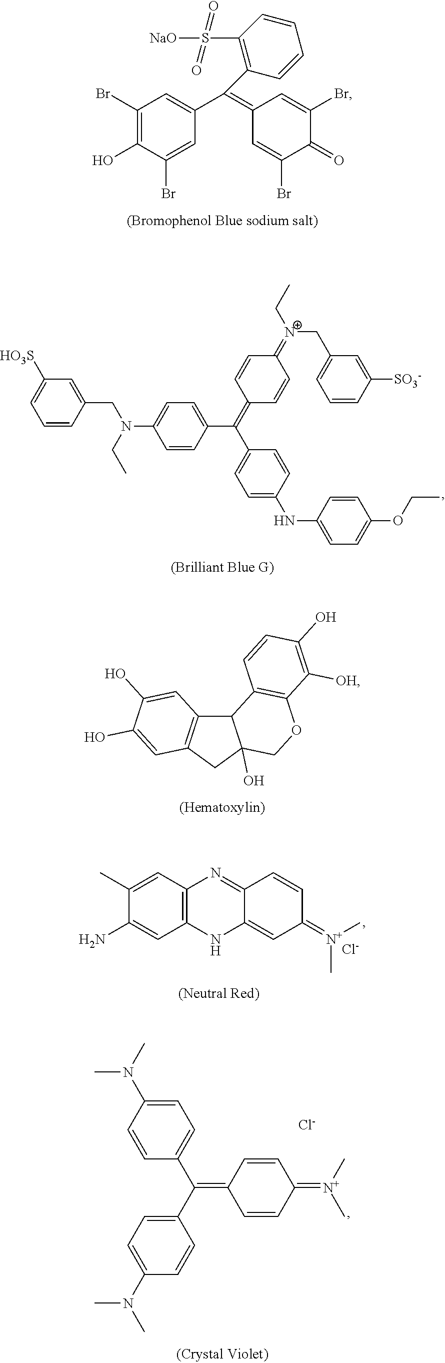

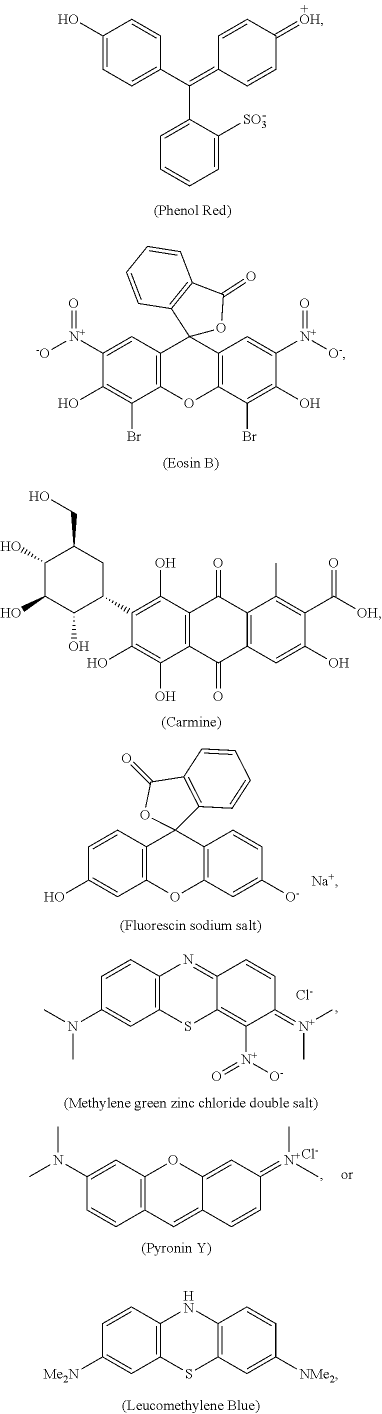

or an acceptable salt, tautomer, reduced form or oxidized form thereof, or any combination or mixture thereof. In some embodiments, the compound is a mixture of safranin O and phenosafranin. In some embodiments, the compound is methylene blue. In some embodiments, the composition comprises ethylenediaminetetraacetic acid (EDTA) in addition to the compound.

In some embodiments, the compound (e.g., a compound of Formula I) has a purity of at least 50%. In some embodiments, the compound has a purity of at least 55%, at least 60%, at least 65%, at least 70%, at least 75%, at least 80%, at least 85%, at least 90%, at least 95%, at least 96%, at least 97%, at least 98%, at least 99%, or at least 99.9%. Methods of determining the purity of a compound are discussed below.

In some embodiments, the composition (e.g., a nucleic acid and/or lipid composition disclosed herein) has a purity of at least 50%. The purity of a composition reflects the amount of components used to make the composition in the composition at any particular point in time. In some embodiments, the composition has a purity of at least 55%, at least 60%, at least 65%, at least 70%, at least 75%, at least 80%, at least 85%, at least 90%, at least 95%, at least 96%, at least 97%, at least 98%, at least 99%, or at least 99.9%.

The purity of a composition may be characterized based on the presence of impurities in the composition at any particular point in time. Impurities include, for instance, lipid-RNA adducts, which are typical degradation products of mRNA-LNPs and elemental metals. In some embodiments, a composition is considered to have an adequate purity if less than 10% of the RNA in a composition is in the form of a lipid-RNA adduct. In some embodiments, a composition is considered to have an adequate purity if less than 9%, 8%, 7%, 6%, 5%, 4%, 3%, 2%, 1%, 0.5%, or 0.1% of the RNA in a composition is in the form of a lipid-RNA adduct.

In some embodiments, the compound (e.g., a compound of Formula I) is free of elemental metals. In some embodiments, the compound contains fewer than 1000 ppm, fewer than 900 ppm, fewer than 800 ppm, fewer than 700 ppm, fewer than 600 ppm, fewer than 500 ppm, fewer than 400 ppm, fewer than 300 ppm, fewer than 200 ppm, fewer than 100 ppm, fewer than 90 ppm, fewer than 80 ppm, fewer than 70 ppm, fewer than 60 ppm, fewer than 50 ppm, fewer than 40 ppm, fewer than 30 ppm, fewer than 20 ppm, fewer than 10 ppm, fewer than 9 ppm, fewer than 8 ppm, fewer than 7 ppm, fewer than 6 ppm, fewer than 5 ppm, fewer than 4 ppm, fewer than 3 ppm, fewer than 2 ppm, fewer than 1 ppm of elemental metals.

According to the present disclosure, the term “elemental metal” is given its ordinary meaning in the art. A metal is an element that readily forms positive ions (i.e., cations) and forms metallic bonds. An elemental metal refers to a metal which is not present in a salt form or otherwise within a compound. Those of ordinary skill in the art will, in general, recognize elemental metals.

Purity can be determined by any suitable method known in the art. Non-limiting examples of methods to determine the purity of a compound include melting point determination, boiling point determination, spectroscopy (e.g., UV-VIS spectroscopy), titration, chromatography (e.g., liquid chromatography or gas chromatography), mass spectroscopy, capillary electrophoresis, and optical rotation.

In some embodiments, the stabilizing compounds disclosed herein are reactive compounds. The term “reactive compound” is given its ordinary meaning in the art. A reactive compound is one with the capacity to undergo a chemical reaction with another compound or with itself. Those of ordinary skill in the art will, in general, recognize compounds that are reactive.

In some embodiments, the reactive compound is a phenothiazinium dye. A phenothiazinium dye is a compound that is closely related to the thiazine-class of heterocyclic compounds, and is a derivative of phenothiazine having the base formula S(C₆H₄)₂NH. Non-limiting examples of phenothiazinium dyes and related compounds include methylene blue (also known as urelene blue, provayblue, proveblue, CI 52015 or basic blue 9), methylene green, thionine, azure A, azure B, toluidine blue O, safranin O, new methylene blue N, acridine orange, proflavine hemisulfate, acriflavine, 1,9-dimethyl-methylene blue, nile blue A, nile red, bromophenol blue, brilliant blue G, hematoxylin, neutral red, crystal violet, phenol red, eosin B, carmine, fluorescein, pyronin Y, and leucomethylene blue (mesylate).

According to some embodiments, compositions disclosed herein are formulated in aqueous solutions. An aqueous solution is a solution in which components are dissolved or otherwise dispersed within water.

In some embodiments, an aqueous solution disclosed herein has a given pH value. In some embodiments, the pH of an aqueous solution disclosed herein is within the range of about 4.5 to about 8.5. In some embodiments, the pH of an aqueous solution is within the range of about 5 to about 8, about 6 to about 8, about 7 to about 8, about 6.5 to about 8, about 6.5 to about 7.5, about 6.5 to about 7, about 7.5 to about 8.5, or any range or combination thereof. In some embodiments, the pH of an aqueous solution is or is about 5, is or is about 5.5, is or is about 6, is or is about 6.5, is or is about 7, is or is about 7.5, or is or is about 8.

In some embodiments, an aqueous solution disclosed herein comprises a pH buffer component, such as a phosphate buffer, a tris buffer, an acetate buffer, a histidine buffer or a citrate buffer, among others. Such a buffer acts to modulate the pH of an aqueous solution, such as an aqueous solution having a pH of 5, 5.5, 6, 6.5, 7, 7.5 or 8.

Aqueous solutions may comprise various concentrations of salts (e.g., sodium chloride, NaCl). In some embodiments, an aqueous solution may comprise a salt (e.g., NaCl) in a concentration of or about 50 mM, of or about 60 mM, of or about 70 mM, of or about 80 mM, of or about 90 mM, of or about 100 mM, of or about 110 mM, of or about 120 mM, of or about 130 mM, of or about 140 mM, of or about 150 mM, of or about 160 mM, of or about 170 mM, of or about 180 mM, of or about 190 mM, of or about 200 mM, or any intermediate concentration therein. In embodiments in which an aqueous solution comprises more than one salt, each salt may independently have a concentration of one or more of the values described above.

According to some aspects of the present disclosure, aqueous solutions (e.g., aqueous solutions comprising nucleic acid, lipid, or nucleic acid and lipid) comprise a compound (e.g., a compound of Formula I) at a concentration of between about 0.1 mM and about 10 mM. In some embodiments, an aqueous solution (e.g., an aqueous solution comprising nucleic acid, lipid, or nucleic acid and lipid) comprises a compound (e.g., a compound of Formula I) at a concentration of between about 0.2 mM and about 10 mM, about 0.3 mM and about 10 mM, about 0.4 mM and about 10 mM, about 0.5 mM and about 10 mM, about 0.6 mM and about 10 mM, about 0.7 mM and about 10 mM, about 0.8 mM and about 10 mM, about 0.9 mM and about 10 mM, about 1 mM and about 10 mM, about 0.5 mM and about 9 mM, about 0.5 mM and about 8 mM, about 0.5 mM and about 7 mM, about 0.5 mM and about 6 mM, about 0.5 mM and about 5 mM, about 0.5 mM and about 4 mM, about 0.5 mM and about 3 mM, about 0.5 mM and about 2 mM, about 0.5 mM and about 1.5 mM, about 0.5 mM and about 1 mM, or any range or combination thereof. In some embodiments, an aqueous solution (e.g., an aqueous solution comprising nucleic acid, lipid, or nucleic acid and lipid) comprises a compound (e.g., a compound of Formula I) at a concentration of or about 0.1 mM, 0.2 mM, 0.3 mM, 0.4 mM, 0.5 mM, 0.6 mM, 0.7 mM, 0.8 mM, 0.9 mM, 1 mM, 1.5 mM, 2 mM, 2.5 mM, 3 mM, 4 mM, 5 mM, 6 mM, 7 mM, 8 mM, 9 mM, or of or about 10 mM. In some embodiments, an aqueous solution (e.g., an aqueous solution comprising nucleic acid, lipid, or nucleic acid and lipid) comprises a compound (e.g., a compound of Formula I) at a concentration of or about 0.5 mM, 1 mM, 1.5 mM, or of or about 2 mM. In some embodiments, an aqueous solution (e.g., an aqueous solution comprising nucleic acid, lipid, or nucleic acid and lipid) does not comprise a compound of Formula I.

According to some aspects of the present disclosure, a composition is a lyophilized product. A lyophilized product is one from which liquid (e.g., water) has been removed by freeze drying, in which a liquid product is frozen and subsequently placed under a vacuum to remove solvent (e.g., water) by sublimation, leaving a composition substantially free of solvent (e.g., water). In some embodiments, a lyophilized product as disclosed herein comprises lipids. In some embodiments, a lyophilized product as disclosed herein comprises lipid nanoparticles. In some embodiments, a lyophilized product as disclosed herein comprises nucleic acid. In some embodiments, a lyophilized product as disclosed herein comprises nucleic acid encapsulated within lipid nanoparticles. In some embodiments, a lyophilized product as disclosed herein comprises a compound of Formula I. In some embodiments, a lyophilized product as disclosed herein comprises methylene blue. In some embodiments, a lyophilized product as disclosed herein comprises lipids, nucleic acids, a compound of Formula I, or any mixture thereof. In some embodiments, a lyophilized product is reconstituted with a solution comprising a compound of Formula I. In some embodiments, a lyophilized product is reconstituted with a solution comprising methylene blue.

According to some aspects, a compound (e.g., a compound of Formula I) permeates into a lipid nanostructure (e.g., lipid nanoparticle, liposome, or lipoplex) as disclosed herein to some extent. Permeation of a compound (e.g., a compound of Formula I) into a lipid nanostructure (e.g., lipid nanoparticle, liposome, or lipoplex) depends on a number of factors, including the lipid composition of the nanostructure, the characteristics of the compound (e.g., charge, hydrophobicity, etc.), the characteristics of the solution in which the nanostructure is comprised (e.g., the pH of the solution, the salt composition of the solution, etc.), and the cargo within the nanostructure (e.g., based on the strength of interaction between the cargo and the compound). Permeation into a lipid nanostructure can be characterized, for example, by a partition coefficient representing the relative concentrations at equilibrium of a compound (e.g., a compound of Formula I) in the lipid nanostructure and in the solution in which the lipid nanostructure is comprised. The partition coefficient is a ratio of concentrations, and therefore represents the relative solubilities of the compound (e.g., a compound of Formula I) in the bulk solution and in the lipid nanostructure. A partition coefficient can be determined by one of skill in the art, for example by equilibrium dialysis.

In some embodiments, permeation of a compound (e.g., a compound of Formula I) into a lipid nanostructure (e.g., lipid nanoparticle, liposome, or lipoplex) as disclosed herein is defined by a partition coefficient K_{LS} representing the partitioning between a solution (e.g., water or an aqueous solution) and the lipid nanostructure comprised within the solution. In some embodiments, the log of the partition coefficient K_{LS} (log K_{LS}) of a compound disclosed herein for a solution disclosed herein and a lipid nanostructure disclosed herein, measured at 25° C. is or is about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10. In some embodiments, the log K_{LS} is defined with reference to a compound (e.g., a compound of Formula I) in water partitioning into a lipid nanostructure disclosed herein. In some embodiments, the log K_{LS} is defined with reference to methylene blue in water partitioning into a lipid nanostructure disclosed herein. In some embodiments, permeation of a compound (e.g., a compound of Formula I) into a lipid nanostructure (e.g., lipid nanoparticle, liposome, or lipoplex) as disclosed herein is defined by a partition coefficient K_{OW} which is defined by the ratio of concentrations of the compound in octanol and water at equilibrium. In some embodiments, the log of the partition coefficient K_{OW} (log K_{OW}) of a compound disclosed herein, measured at 25° C. is or is about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10. In some embodiments, log K_{OW} of a compound disclosed herein, measured at 25° C., is or is about 6. In some embodiments, log K_{OW} of a compound disclosed herein, measured at 25° C., is or is about 5.85. In some embodiments, log K_{OW} of a compound disclosed herein, measured at 25° C., is or is about 5.

In some embodiments, permeation of a compound (e.g., a compound of Formula I) into a lipid nanostructure (e.g., lipid nanoparticle, liposome, or lipoplex) as disclosed herein is defined by the amount of the compound (e.g., by weight) present in the lipid nanostructure following incubation of the lipid nanostructure with a given concentration of the compound. In some embodiments, following incubation of a lipid nanostructure disclosed herein with a 1 mM solution of a compound disclosed herein, the lipid nanostructure comprises 0.001%, 0.005%, 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%, 1%, 1.1%, 1.12%, 1.14%, 1.16%, 1.18%, 1.2%, 1.22%, 1.24%, 1.26%, 1.28%, 1.3%, 1.32%, 1.34%, 1.36%, 1.38%, 1.4%, 1.42%, 1.44%, 1.46%, 1.48%, 1.5%, 1.52%, 1.54%, 1.56%, 1.58%, 1.6%, 1.62%, 1.64%, 1.66%, 1.68%, 1.7%, 1.72%, 1.74%, 1.76%, 1.78%, 1.8%, 1.82%, 1.84%, 1.86%, 1.88%, 1.9%, 1.92%, 1.94%, 1.96%, 1.98%, 2%, 2.2%, 2.4%, 2.6%, 2.8%, 3% by weight of the compound, or any range or combination thereof.

According to some aspects, a compound (e.g., a compound of Formula I) disclosed herein is cationic. In some embodiments, a compound disclosed herein is cationic and has a charge of +1, +2, +3, or +4. In some embodiments, the compound has a charge of +1. In some embodiments, the compound has a charge of +2.

In some embodiments, a compound disclosed herein is water soluble. In some embodiments, the compound has a solubility in water of at least 10 mg/L (e.g., at least 100 mg/L, at least 200 mg/L, at least 300 mg/L, at least 400 mg/L, at least 500 mg/L, at least 600 mg/L, at least 700 mg/L, at least 800 mg/L, at least 900 mg/L, at least 1 g/L, at least 2 g/L, at least 3 g/L, at least 10 g/L, or more) at 25° C. In some embodiments, the compound has a solubility in water of or about 50 g/L at 25° C. In some embodiments, the compound has a solubility in water of or about 45 g/L at 25° C. In some embodiments, the compound has a solubility in water of or about 43.6 g/L at 25° C.

According to some aspects, a compound (e.g., a compound of Formula I) disclosed herein has a low cytotoxicity. In some embodiments, a compound disclosed herein has a cytotoxicity LC50 value of at least 5 mg/L (e.g., at least 10 mg/L, at least 15 mg/L, at least 20 mg/L, at least 25 mg/L, at least 30 mg/L, at least 35 mg/L, at least 40 mg/L, at least 45 mg/L, at least 50 mg/L, or more) when measured in mammalian cells (e.g., human cells or murine cells) in culture, or when measured in test organisms (e.g., fish, such as zebrafish or Mystus vittatus).

The stabilizing compounds may be used together with therapeutic agents, such as nucleic acids, lipid formulations or combinations thereof.

As disclosed herein, the term “nucleic acid” refers to multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G))). As used herein, the term nucleic acid refers to polyribonucleotides as well as polydeoxyribonucleotides. The term nucleic acid shall also include polynucleosides (i.e., a polynucleotide minus the phosphate) and any other organic base containing polymer. Non-limiting examples of nucleic acids include chromosomes, genomic loci, genes or gene segments that encode polynucleotides or polypeptides, coding sequences, non-coding sequences (e.g., intron, 5′-UTR, or 3′-UTR) of a gene, pri-mRNA, pre-mRNA, cDNA, mRNA, etc. In some embodiments, the nucleic acid is mRNA. A nucleic acid may include a substitution and/or modification. In some embodiments, the substitution and/or modification is in one or more bases and/or sugars. For example, in some embodiments a nucleic acid includes nucleic acids having backbone sugars that are covalently attached to low molecular weight organic groups other than a hydroxyl group at the 2′ position and other than a phosphate group or hydroxy group at the 5′ position. Thus, in some embodiments, a substituted or modified nucleic acid includes a 2′-O-alkylated ribose group. In some embodiments, a modified nucleic acid includes sugars such as hexose, 2′-F hexose, 2′-amino ribose, constrained ethyl (cEt), locked nucleic acid (LNA), arabinose or 2′-fluoroarabinose instead of ribose. Thus, in some embodiments, a nucleic acid is heterogeneous in backbone composition thereby containing any possible combination of polymer units linked together such as peptide-nucleic acids (which have an amino acid backbone with nucleic acid bases).

In some embodiments, a nucleic acid is DNA, RNA, PNA, cEt, LNA, ENA or hybrids including any chemical or natural modification thereof. Chemical and natural modifications are well known in the art. Non-limiting examples of modifications include modifications designed to increase translation of the nucleic acid, to increase cell penetration or sub-cellular distribution of the nucleic acid, to stabilize the nucleic acid against nucleases and other enzymes that degrade or interfere with the structure or activity of the nucleic acid, and to improve the pharmacokinetic properties of the nucleic acid.

In some embodiments, the compositions of the present disclosure comprise a RNA having an open reading frame (ORF) encoding a polypeptide. In some embodiments, the RNA is a messenger RNA (mRNA). In some embodiments, the RNA (e.g., mRNA) further comprises a 5′ UTR, 3′ UTR, a poly(A) tail and/or a 5′ cap analog.

Messenger RNA (mRNA) is any RNA that encodes a (at least one) protein (a naturally-occurring, non-naturally-occurring, or modified polymer of amino acids) and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents RNA (e.g., mRNA), the “T”s would be substituted for “U”s. Thus, any of the DNAs disclosed and identified by a particular sequence identification number herein also disclose the corresponding RNA (e.g., mRNA) sequence complementary to the DNA, where each “T” of the DNA sequence is substituted with “U.”

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and 3′ UTRs, but that those elements, unlike the ORF, need not necessarily be present in an RNA polynucleotide of the present disclosure.

Naturally-occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, a composition includes an RNA polynucleotide having an open reading frame encoding at least one polypeptide having at least one modification, at least one 5′ terminal cap, and is formulated within a lipid nanoparticle along with the stabilizing compound. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

The 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments, a composition comprises an RNA (e.g., mRNA) having an ORF that encodes a signal peptide fused to the expressed polypeptide. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. A signal peptide may have a length of 15-60 amino acids.

In some embodiments, an ORF encoding a polypeptide is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park Calif.) and/or proprietary methods. In some embodiments, the open reading frame (ORF) sequence is optimized using optimization algorithms.

In some embodiments, an RNA (e.g., mRNA) is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

The compositions of the present disclosure comprise, in some embodiments, an RNA having an open reading frame encoding a polypeptide, wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

In some embodiments, modified nucleobases in nucleic acids (e.g., RNA nucleic acids, such as mRNA nucleic acids) comprise 1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a mRNA of the disclosure comprises 1-methyl-pseudouridine (m1ψ) substitutions at one or more or all uridine positions of the nucleic acid.

In some embodiments, a mRNA of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a mRNA of the disclosure comprises pseudouridine (w) substitutions at one or more or all uridine positions of the nucleic acid.

In some embodiments, a mRNA of the disclosure comprises pseudouridine (w) substitutions at one or more or all uridine positions of the nucleic acid and 5-methyl cytidine substitutions at one or more or all cytidine positions of the nucleic acid.

In some embodiments, a mRNA of the disclosure comprises uridine at one or more or all uridine positions of the nucleic acid.

In some embodiments, mRNAs are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the poly(A) tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The mRNAs of the present disclosure may comprise one or more regions or parts which act or function as an untranslated region. Where mRNAs are designed to encode at least one polypeptide of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas, the 3′ UTR starts immediately following the stop codon and continues until the transcriptional termination signal. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

According to some aspects, a compound (e.g., a compound of Formula I) disclosed herein interacts with a nucleic acid. In some embodiments, a compound disclosed herein interacts with a nucleic acid comprised within a lipid nanostructure (e.g., a lipid nanoparticle, liposome, or lipoplex) disclosed herein. In some embodiments, a compound disclosed herein intercalates with a nucleic acid. In some embodiments, a compound disclosed herein intercalates with a nucleic acid comprised within a lipid nanostructure. In some embodiments, a compound disclosed herein binds with a nucleic acid. In some embodiments, a compound disclosed herein reversibly binds with a nucleic acid. In some embodiments, a compound disclosed herein binds with a nucleic acid comprised within a lipid nanostructure.

In some embodiments, a compound (e.g., a compound of Formula I) disclosed herein interacts with a nucleic acid (e.g., an mRNA) with a binding affinity defined by a particular equilibrium dissociation constant. In some embodiments, the equilibrium dissociation constant is less than 10⁻³ M (e.g., less than 10⁻⁴ M, less than 10⁻⁵ M, less than 10⁻⁵ M, less than 10⁻⁷ M, less than 10⁻⁸ M, or less than 10⁻⁹ M). In some embodiments, the equilibrium dissociation constant is between 10⁻³ M and 10⁻⁴ M, between 10⁻³ M and 10⁻⁵ M, between 10⁻³ M and 10⁻⁶ M, between 10⁻³ M and 10⁻⁷ M, between 10⁻³ M and 10⁻⁸ M, between 10⁻³ M and 10⁻⁹ M, between 10⁻³ M and 10⁻¹⁰ M, between 10⁻⁴ M and 10⁻⁵ M, between 10⁻⁴ M and 10⁻⁶ M, between 10⁻⁴ M and 10⁻⁷ M, between 10⁻⁴ M and 10⁻⁸ M, between 10⁻⁴ M and 10⁻⁹ M, between 10⁻⁴ M and 10⁻¹⁰ M, between 10⁻⁵ M and 10⁻⁶ M, between 10⁻⁵ M and 10⁻⁷ M, between 10⁻⁵ M and 10⁻⁸ M, between 10⁻⁵ M and 10⁻⁹ M, between 10⁻⁵ M and 10⁻¹⁰ M, between 10⁻⁶ M and 10⁻⁷ M, between 10⁻⁶ M and 10⁻⁸ M, between 10⁻⁶ M and 10⁻⁹ M, between 10⁻⁶ M and 10⁻¹⁰ M, between 10⁻⁷ M and 10⁻⁸ M, between 10⁻⁷ M and 10⁻⁹ M, between 10⁻⁷ M and 10⁻¹⁰ M, between 10⁻⁸ M and 10⁻⁹ M, between 10⁻⁸ M and 10⁻¹⁰ M, or between 10⁻⁹ M and 10⁻¹⁰ M. In some embodiments, the equilibrium dissociation constant is between 10⁻³ M and 10⁻⁴ M or between 10⁻³ M and 10⁻⁵ M.

In some embodiments, a compound (e.g., a compound of Formula I) disclosed herein confers increased stability to a nucleic acid (e.g., an mRNA) in a folded structure. In some embodiments, a compound disclosed herein confers increased stability to a folded structure of a nucleic acid (e.g., an mRNA) relative to its unfolded or less folded (i.e., more linear) form. Changes in stability of a folded structure of a nucleic acid can be identified by one of ordinary skill in the art, for example, by circular dichroism. Such changes in stability of a folded structure may, for example, result in changes in the amplitude of peaks in circular dichroism spectra. In some embodiments, a compound disclosed herein enhances the thermal stability of a nucleic acid (e.g., an mRNA) in a folded state. Changes in thermal stability of a folded state of a nucleic acid can be identified by one of ordinary skill in the art, for example, by differential scanning calorimetry. Such changes in thermal stability may, for example, result in shifts of differential scanning calorimetry thermograms.

In some embodiments, a compound (e.g., a compound of Formula I) disclosed herein causes compaction of a nucleic acid molecule (e.g., an mRNA) upon interacting with the nucleic acid molecule. In some embodiments, a compound disclosed herein causes a decrease in the hydrodynamic radius of a nucleic acid molecule (e.g. an mRNA) upon interaction with the nucleic acid molecule. In some embodiments, a compound disclosed herein causes compaction or a decrease in the hydrodynamic radius of a nucleic acid molecule by 5%, 10%, 15%, 20%, 25%, 30%, 35%, 40%, 45%, 50%, 55%, 60%, or more. In some embodiments, a compound disclosed herein causes compaction or a decrease in the hydrodynamic radius of a nucleic acid molecule when the compound is in a concentration of 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM, 50 μM, 60 μM, 70 μM, 80 μM, 90 μM, or 100 μM. In some embodiments, a compound disclosed herein causes compaction or a decrease in the hydrodynamic radius of a nucleic acid molecule when the compound is in a concentration of 10 μM. In some embodiments, a compound (e.g., a compound of Formula I) disclosed herein causes compaction of a nucleic acid molecule (e.g., an mRNA) within a lipid nanostructure (e.g., a lipid nanoparticle, liposome, or lipoplex) disclosed herein. In some embodiments, a compound disclosed herein causes compaction of a nucleic acid molecule within a lipid nanostructure without changing the size of the lipid nanostructure. Compaction of a nucleic acid molecule or a decrease in its hydrodynamic radius can be measured by one of ordinary skill in the art, for example, via dynamic light scattering or transmission electron microscopy measurements.

### Lipid Nanoparticle Formulations

In some embodiments, nucleic acids of the invention are formulated as lipid nanoparticle (LNP) compositions. Lipid nanoparticles typically comprise amino lipid, phospholipid, structural lipid and PEG lipid components along with the nucleic acid cargo of interest. The lipid nanoparticles of the invention can be generated using components, compositions, and methods as are generally known in the art, see for example PCT/US2016/052352; PCT/US2016/068300; PCT/US2017/037551; PCT/US2015/027400; PCT/US2016/047406; PCT/US2016/000129; PCT/US2016/014280; PCT/US2016/014280; PCT/US2017/038426; PCT/US2014/027077; PCT/US2014/055394; PCT/US2016/052117; PCT/US2012/069610; PCT/US2017/027492; PCT/US2016/059575; PCT/US2016/069491; PCT/US2016/069493; and PCT/US2014/066242, all of which are incorporated by reference herein in their entirety.

In some embodiments, the lipid nanoparticle comprises a molar ratio of 20-60% amino lipid relative to the other lipid components. For example, the lipid nanoparticle may comprise a molar ratio of 20-50%, 20-40%, 20-30%, 30-60%, 30-50%, 30-40%, 40-60%, 40-50%, or 50-60% amino lipid. In some embodiments, the lipid nanoparticle comprises a molar ratio of 20%, 30%, 40%, 50, or 60% amino lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-lipid.

In some embodiments, the lipid nanoparticle comprises a molar ratio of 20-60% amino lipid, 5-25% phospholipid, 25-55% structural lipid, and 0.5-15% PEG lipid.

In some embodiments, the lipid nanoparticle comprises a molar ratio of 20-60% amino lipid, 5-30% phospholipid, 10-55% structural lipid, and 0.5-15% PEG lipid.

Amino Lipids

In some aspects, the amino lipids of the present disclosure may be one or more of compounds of Formula (IV):

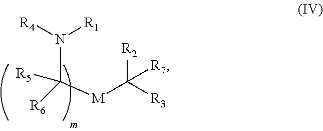

or their N-oxides, or salts or isomers thereof, wherein:

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of hydrogen, a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —N(R)S(O)₂R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, C₁, Br, and I; and

m is selected from 5, 6, 7, 8, 9, 10, 11, 12, and 13; and wherein when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In certain embodiments, a subset of compounds of Formula (IV) includes those of Formula (IV-A):

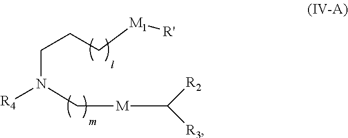

or its N-oxide, or a salt or isomer thereof, wherein 1 is selected from 1, 2, 3, 4, and 5; m is selected from 5, 6, 7, 8, and 9; M₁ is a bond or M′; R₄ is hydrogen, unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (IV) includes those of Formula (IV-B):

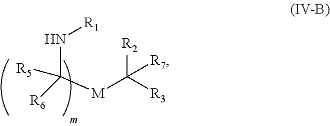

or its N-oxide, or a salt or isomer thereof in which all variables are as defined herein. For example, in is selected from 5, 6, 7, 8, and 9; R₄ is hydrogen, unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, in is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (IV) includes those of Formula (V):

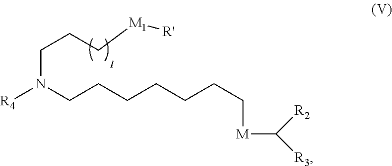

or its N-oxide, or a salt or isomer thereof, wherein 1 is selected from 1, 2, 3, 4, and 5; M₁ is a bond or M′; R₄ is hydrogen, unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In one embodiment, the compounds of Formula (IV) are of Formula (Va)

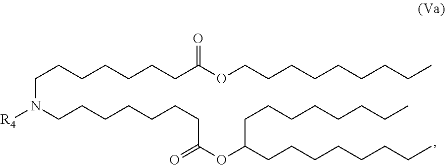

or their N-oxides, or salts or isomers thereof, wherein R₄ is as described herein.

In another embodiment, the compounds of Formula (IV) are of Formula (Vb),

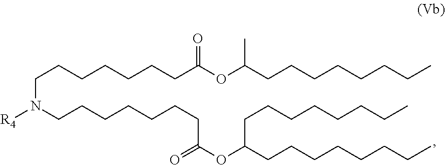

or their N-oxides, or salts or isomers thereof, wherein R₄ is as described herein.

In another embodiment, the compounds of Formula (IV) are of Formula (Vc) or (Ve):

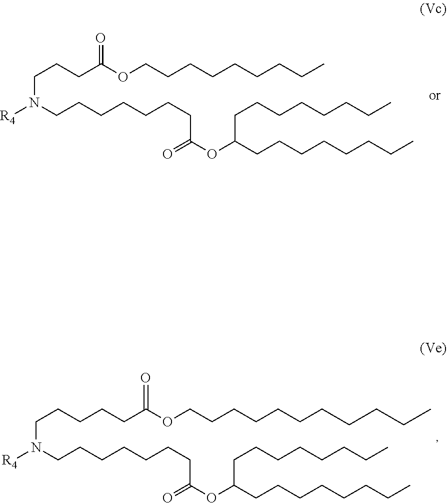

or their N-oxides, or salts or isomers thereof, wherein R₄ is as described herein.

In another embodiment, the compounds of Formula (IV) are of Formula (Vf):

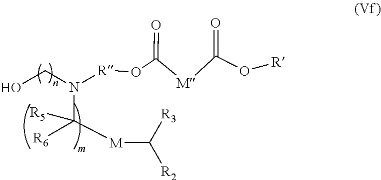

or their N-oxides, or salts or isomers thereof,

wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In a further embodiment, the compounds of Formula (IV) are of Formula (Vd),

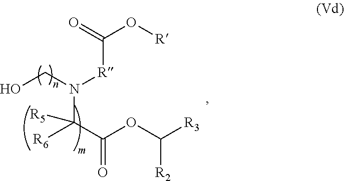

or their N-oxides, or salts or isomers thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In a further embodiment, the compounds of Formula (IV) are of Formula (Vg),

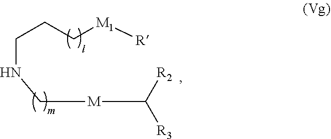

or their N-oxides, or salts or isomers thereof, wherein 1 is selected from 1, 2, 3, 4, and 5; m is selected from 5, 6, 7, 8, and 9; M₁ is a bond or M′; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R₂ and R₃ are independently selected from the group consisting of C₅₋₁₄ alkyl and C₅₋₁₄ alkenyl.

In some embodiments, the amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

In some embodiments, the amino lipid is

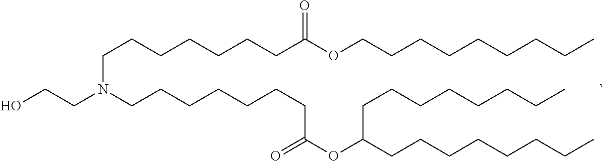 [SMILES: CCCCCCCCCOC(=O)CCCCCCCN(CCO)CCCCCCCC(=O)OC(CCCCCCCC)CCCCCCCC]

or a salt thereof.

In some embodiments, the amino lipid is

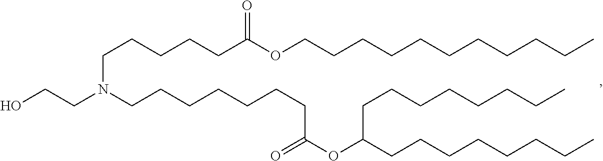 [SMILES: CCCCCCCCCCCOC(=O)CCCCCN(CCO)CCCCCCCC(=O)OC(CCCCCCCC)CCCCCCCC]

or a salt thereof.

The central amine moiety of a lipid according to Formula (IV), (IV-A), (IV-B), (V), (Va), (Vb), (Vc), (Vd), (Ve), (Vf), or (Vg) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

In some aspects, the amino lipids of the present disclosure may be one or more of compounds of formula (VI),

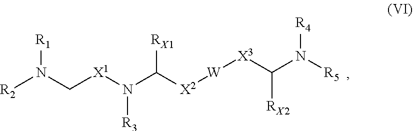

or salts or isomers thereof, wherein

W is

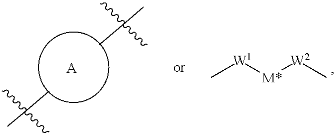

ring A is

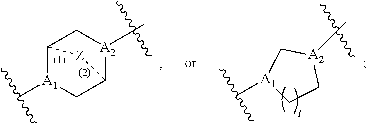

t is 1 or 2;

A₁ and A₂ are each independently selected from CH or N;

Z is CH₂ or absent wherein when Z is CH₂, the dashed lines (1) and (2) each represent a single bond; and when Z is absent, the dashed lines (1) and (2) are both absent;

R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;

R_{X1} and R_{X2} are each independently H or C₁₋₃ alkyl;

each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;

M* is C₁-C₆ alkyl,

W¹ and W² are each independently selected from the group consisting of —O— and —N(R₆)—;

each R₆ is independently selected from the group consisting of H and C₁₋₅ alkyl;

X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;

each Y is independently a C₃₋₆ carbocycle;

each R* is independently selected from the group consisting of C₁₋₁₂ alkyl and C₂₋₁₂ alkenyl;

each R is independently selected from the group consisting of C₁₋₃ alkyl and a C₃₋₆ carbocycle;

each R′ is independently selected from the group consisting of C₁₋₁₂ alkyl, C₂₋₁₂ alkenyl, and H;

each R″ is independently selected from the group consisting of C₃₋₁₂ alkyl, C₃₋₁₂ alkenyl and —R*MR′; and

n is an integer from 1-6;

wherein when ring A is

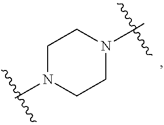 [SMILES: *N1CCN(*)CC1]

then

i) at least one of X¹, X², and X³ is not —CH₂—; and/or

ii) at least one of R₁, R₂, R₃, R₄, and R₅ is —R″MR′.

In some embodiments, the compound is of any of formulae (VIa1)-(VIa8):

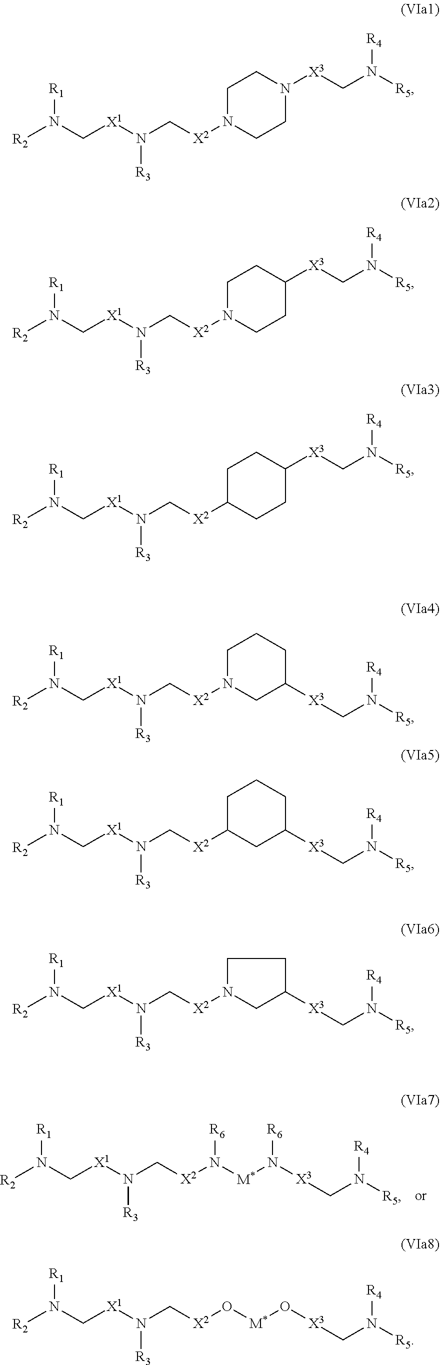

In some embodiments, the amino lipid is

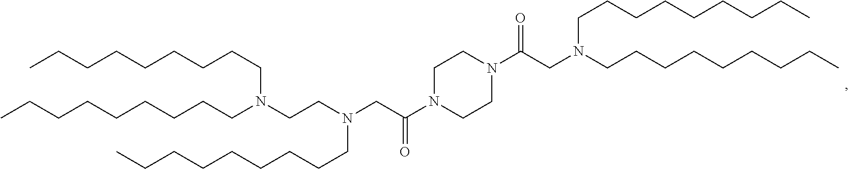 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (VI), (VIa1), (VIa2), (VIa3), (VIa4), (VIa5), (VIa6), (VIa7), or (VIa8) may be protonated at a physiological pH. Thus, a lipid may have a positive or partial positive charge at physiological pH.

Phospholipids

The lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In certain embodiments, a phospholipid useful or potentially useful in the present invention is an analog or variant of DSPC. In certain embodiments, a phospholipid useful or potentially useful in the present invention is a compound of Formula (VII):

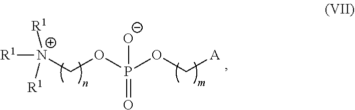

or a salt thereof, wherein:

each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;

n is 1, 2, 3, 4, 5, 6, 7, 8, 9, or 10;

m is 0, 1, 2, 3, 4, 5, 6, 7, 8, 9, or 10;

A is of the formula:

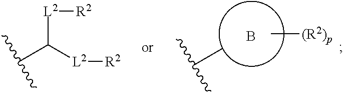

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2;

provided that the compound is not of the formula:

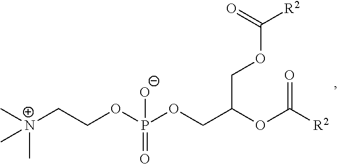

wherein each instance of R² is independently unsubstituted alkyl, unsubstituted alkenyl, or unsubstituted alkynyl.

In some embodiments, the phospholipids may be one or more of the phospholipids described in PCT Application No. PCT/US2018/037922.

Structural Lipids

The lipid composition of a pharmaceutical composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

Polyethylene Glycol (PEG)-Lipids

The lipid composition of a pharmaceutical composition disclosed herein can comprise one or more polyethylene glycol (PEG) lipids.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

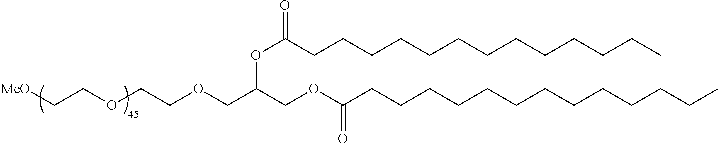

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (V). Provided herein are compounds of Formula (VIII):

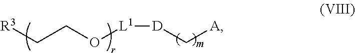

or salts thereof, wherein:

R³ is —OR^{O};

R^{O} is hydrogen, optionally substituted alkyl, or an oxygen protecting group;

r is an integer between 1 and 100, inclusive;

L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

D is a moiety obtained by click chemistry or a moiety cleavable under physiological conditions;

m is 0, 1, 2, 3, 4, 5, 6, 7, 8, 9, or 10;

A is of the formula:

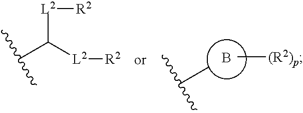

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2.

In certain embodiments, the compound of Formula (VIII) is a PEG-OH lipid (i.e., R³ is —OR^{O}, and R^{O} is hydrogen). In certain embodiments, the compound of Formula (VIII) is of Formula (VIII-OH):

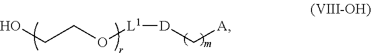

or a salt thereof.

In certain embodiments, a PEG lipid useful in the present invention is a PEGylated fatty acid. In certain embodiments, a PEG lipid useful in the present invention is a compound of Formula (IX). Provided herein are compounds of Formula (IX):

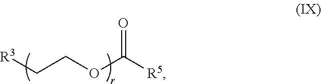

or a salts thereof, wherein:

R³ is —OR^{O};

R^{O} is hydrogen, optionally substituted alkyl or an oxygen protecting group;

r is an integer between 1 and 100, inclusive;

R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R′ are replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, S(O)₂₀, OS(O)₂₀, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O; and

each instance of R^{N} is independently hydrogen, optionally substituted alkyl, or a nitrogen protecting group.

In certain embodiments, the compound of Formula (IX) is of Formula (IX-OH):

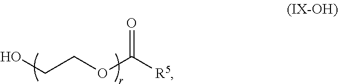

or a salt thereof. In some embodiments, r is 40-50.

In yet other embodiments the compound of Formula (IX) is:

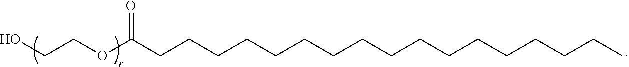

or a salt thereof.

In one embodiment, the compound of Formula (IX) is

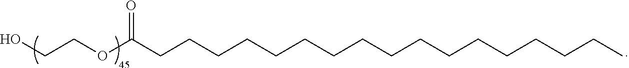

In some aspects, the lipid composition of the pharmaceutical compositions disclosed herein does not comprise a PEG-lipid.

In some embodiments, the PEG-lipids may be one or more of the PEG lipids described in U.S. Application No. U.S. Ser. No. 15/674,872.

In some embodiments, a LNP of the invention comprises an amino lipid of any of Formula IV, V or VI, a phospholipid comprising DSPC, a structural lipid, and a PEG lipid comprising PEG-DMG.

In some embodiments, a LNP of the invention comprises an amino lipid of any of Formula IV, V or VI, a phospholipid comprising DSPC, a structural lipid, and a PEG lipid comprising a compound having Formula IX.

In some embodiments, a LNP of the invention comprises an amino lipid of Formula IV, V or VI, a phospholipid comprising a compound having Formula VII, a structural lipid, and the PEG lipid comprising a compound having Formula VIII or IX.

In some embodiments, a LNP of the invention comprises an amino lipid of Formula IV, V or VI, a phospholipid comprising a compound having Formula VII, a structural lipid, and the PEG lipid comprising a compound having Formula VIII or IX.

In some embodiments, a LNP of the invention comprises an amino lipid of Formula IV, V or VI, a phospholipid having Formula VII, a structural lipid, and a PEG lipid comprising a compound having Formula IX.

In some embodiments, a LNP of the invention comprises an N:P ratio of from about 2:1 to about 30:1.

In some embodiments, a LNP of the invention comprises an N:P ratio of about 6:1.

In some embodiments, a LNP of the invention comprises an N:P ratio of about 3:1, 4:1, or 5:1.

In some embodiments, a LNP of the invention comprises a wt/wt ratio of the amino lipid component to the RNA of from about 10:1 to about 100:1.

In some embodiments, a LNP of the invention comprises a wt/wt ratio of the amino lipid component to the RNA of about 20:1.

In some embodiments, a LNP of the invention comprises a wt/wt ratio of the amino lipid component to the RNA of about 10:1.

In some embodiments, a LNP of the invention has a mean diameter from about 30 nm to about 150 nm.

In some embodiments, a LNP of the invention has a mean diameter from about 60 nm to about 120 nm.

As disclosed herein, a lipid nanoparticle (LNP) refers to a nanoscale construct (e.g., a nanoparticle, typically less than 100 nm in diameter) comprising lipid molecules arranged in a substantially spherical (i.e., spheroid) geometry, sometimes encapsulating one or more additional molecular species. A LNP may comprise or one or more types of lipids, including but not limited to amino lipids (e.g., ionizable amino lipids), neutral lipids, non-cationic lipids, charged lipids, PEG-modified lipids, phospholipids, structural lipids and sterols. In some embodiments, a LNP may further comprise one or more cargo molecules, including but not limited to nucleic acids (e.g., mRNA, plasmid DNA, DNA or RNA oligonucleotides, siRNA, shRNA, snRNA, snoRNA, lncRNA, etc.), small molecules, proteins and peptides. A LNP may have a unilamellar structure (i.e., having a single lipid layer or lipid bilayer surrounding a central region) or a multilamellar structure (i.e., having more than one lipid layer or lipid bilayer surrounding a central region). In some embodiments, a lipid nanoparticle may be a liposome. A liposome is a nanoparticle comprising lipids arranged into one or more concentric lipid bilayers around a central region. The central region of a liposome may comprises an aqueous solution, suspension, or other aqueous composition.

In some embodiments, a lipid nanoparticle may comprise two or more components (e.g., amino lipid and nucleic acid, PEG-lipid, phospholipid, structural lipid). For instance, a lipid nanoparticle may comprise an amino lipid and a nucleic acid. Compositions comprising the lipid nanoparticles, such as those described herein, may be used for a wide variety of applications, including the stealth delivery of therapeutic payloads with minimal adverse innate immune response.

Effective in vivo delivery of nucleic acids represents a continuing medical challenge. Exogenous nucleic acids (i.e., originating from outside of a cell or organism) are readily degraded in the body, e.g., by the immune system. Accordingly, effective delivery of nucleic acids to cells often requires the use of a particulate carrier (e.g., lipid nanoparticles). The particulate carrier should be formulated to have minimal particle aggregation, be relatively stable prior to intracellular delivery, effectively deliver nucleic acids intracellularly, and illicit no or minimal immune response. To achieve minimal particle aggregation and pre-delivery stability, many conventional particulate carriers have relied on the presence and/or concentration of certain components (e.g., PEG-lipid). However, it has been discovered that certain components may decrease the stability of encapsulated nucleic acids (e.g., mRNA molecules). The reduced stability may limit the broad applicability of the particulate carriers. As such, there remains a need for methods by which to improve the stability of nucleic acid (e.g., mRNA) encapsulated within lipid nanoparticles.

In some embodiments, the lipid nanoparticles comprise one or more of ionizable molecules, polynucleotides, and optional components, such as structural lipids, sterols, neutral lipids, phospholipids and a molecule capable of reducing particle aggregation (e.g., polyethylene glycol (PEG), PEG-modified lipid), such as those described above.

In some embodiments, a LNP described herein may include one or more ionizable molecules (e.g., amino lipids or ionizable lipids). The ionizable molecule may comprise a charged group and may have a certain pKa. In certain embodiments, the pKa of the ionizable molecule may be greater than or equal to about 6, greater than or equal to about 6.2, greater than or equal to about 6.5, greater than or equal to about 6.8, greater than or equal to about 7, greater than or equal to about 7.2, greater than or equal to about 7.5, greater than or equal to about 7.8, greater than or equal to about 8. In some embodiments, the pKa of the ionizable molecule may be less than or equal to about 10, less than or equal to about 9.8, less than or equal to about 9.5, less than or equal to about 9.2, less than or equal to about 9.0, less than or equal to about 8.8, or less than or equal to about 8.5. Combinations of the above referenced ranges are also possible (e.g., greater than or equal to 6 and less than or equal to about 8.5). Other ranges are also possible. In embodiments in which more than one type of ionizable molecule are present in a particle, each type of ionizable molecule may independently have a pKa in one or more of the ranges described above.

In general, an ionizable molecule comprises one or more charged groups. In some embodiments, an ionizable molecule may be positively charged or negatively charged. For instance, an ionizable molecule may be positively charged. For example, an ionizable molecule may comprise an amine group. As used herein, the term “ionizable molecule” has its ordinary meaning in the art and may refer to a molecule or matrix comprising one or more charged moiety. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule and/or matrix may be selected as desired.

In some cases, an ionizable molecule (e.g., an amino lipid or ionizable lipid) may include one or more precursor moieties that can be converted to charged moieties. For instance, the ionizable molecule may include a neutral moiety that can be hydrolyzed to form a charged moiety, such as those described above. As a non-limiting specific example, the molecule or matrix may include an amide, which can be hydrolyzed to form an amine, respectively. Those of ordinary skill in the art will be able to determine whether a given chemical moiety carries a formal electronic charge (for example, by inspection, pH titration, ionic conductivity measurements, etc.), and/or whether a given chemical moiety can be reacted (e.g., hydrolyzed) to form a chemical moiety that carries a formal electronic charge.

The ionizable molecule (e.g., amino lipid or ionizable lipid) may have any suitable molecular weight. In certain embodiments, the molecular weight of an ionizable molecule is less than or equal to about 2,500 g/mol, less than or equal to about 2,000 g/mol, less than or equal to about 1,500 g/mol, less than or equal to about 1,250 g/mol, less than or equal to about 1,000 g/mol, less than or equal to about 900 g/mol, less than or equal to about 800 g/mol, less than or equal to about 700 g/mol, less than or equal to about 600 g/mol, less than or equal to about 500 g/mol, less than or equal to about 400 g/mol, less than or equal to about 300 g/mol, less than or equal to about 200 g/mol, or less than or equal to about 100 g/mol. In some instances, the molecular weight of an ionizable molecule is greater than or equal to about 100 g/mol, greater than or equal to about 200 g/mol, greater than or equal to about 300 g/mol, greater than or equal to about 400 g/mol, greater than or equal to about 500 g/mol, greater than or equal to about 600 g/mol, greater than or equal to about 700 g/mol, greater than or equal to about 1000 g/mol, greater than or equal to about 1,250 g/mol, greater than or equal to about 1,500 g/mol, greater than or equal to about 1,750 g/mol, greater than or equal to about 2,000 g/mol, or greater than or equal to about 2,250 g/mol. Combinations of the above ranges (e.g., at least about 200 g/mol and less than or equal to about 2,500 g/mol) are also possible. In embodiments in which more than one type of ionizable molecules are present in a particle, each type of ionizable molecule may independently have a molecular weight in one or more of the ranges described above.

In some embodiments, the percentage (e.g., by weight, or by mole) of a single type of ionizable molecule (e.g., amino lipid or ionizable lipid) and/or of all the ionizable molecules within a particle may be greater than or equal to about 15%, greater than or equal to about 16%, greater than or equal to about 17%, greater than or equal to about 18%, greater than or equal to about 19%, greater than or equal to about 20%, greater than or equal to about 21%, greater than or equal to about 22%, greater than or equal to about 23%, greater than or equal to about 24%, greater than or equal to about 25%, greater than or equal to about 30%, greater than or equal to about 35%, greater than or equal to about 40%, greater than or equal to about 42%, greater than or equal to about 45%, greater than or equal to about 48%, greater than or equal to about 50%, greater than or equal to about 52%, greater than or equal to about 55%, greater than or equal to about 58%, greater than or equal to about 60%, greater than or equal to about 62%, greater than or equal to about 65%, or greater than or equal to about 68%. In some instances, the percentage (e.g., by weight, or by mole) may be less than or equal to about 70%, less than or equal to about 68%, less than or equal to about 65%, less than or equal to about 62%, less than or equal to about 60%, less than or equal to about 58%, less than or equal to about 55%, less than or equal to about 52%, less than or equal to about 50%, or less than or equal to about 48%. Combinations of the above referenced ranges are also possible (e.g., greater than or equal to 20% and less than or equal to about 60%, greater than or equal to 40% and less than or equal to about 55%, etc.). In embodiments in which more than one type of ionizable molecule is present in a particle, each type of ionizable molecule may independently have a percentage (e.g., by weight, or by mole) in one or more of the ranges described above. The percentage (e.g., by weight, or by mole) may be determined by extracting the ionizable molecule(s) from the dried particles using, e.g., organic solvents, and measuring the quantity of the agent using high pressure liquid chromatography (i.e., HPLC), liquid chromatography-mass spectrometry (LC-MS), nuclear magnetic resonance (NMR), or mass spectrometry (MS). Those of ordinary skill in the art would be knowledgeable of techniques to determine the quantity of a component using the above-referenced techniques. For example, HPLC may be used to quantify the amount of a component, by, e.g., comparing the area under the curve of a HPLC chromatogram to a standard curve.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

Some aspects of the present disclosure provide lipid compositions comprising a lipid and a compound of compound of Formula I:

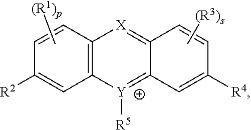

or an acceptable salt, tautomer, reduced form or oxidized form thereof. R¹, R², R³, R⁴, R⁵, X, Y, p, and s are as described above.

According to the disclosures herein, a lipid composition may comprise one or more lipids as described herein. Such lipids may include those useful in the preparation of lipid nanoparticle formulations as described above or as known in the art.

In some embodiments, a subject to which a composition comprising a nucleic acid, a lipid, and/or a compound of Formula I is administered is a subject that suffers from or is at risk of suffering from a disease, disorder or condition, including a communicable or non-communicable disease, disorder or condition. As used herein, “treating” a subject can include either therapeutic use or prophylactic use relating to a disease, disorder or condition, and may be used to describe uses for the alleviation of symptoms of a disease, disorder or condition, uses for vaccination against a disease, disorder or condition, and uses for decreasing the contagiousness of a disease, disorder or condition, among other uses.

In some embodiments the nucleic acid is an mRNA vaccine designed to achieve particular biologic effects. Exemplary vaccines of the invention feature mRNAs encoding a particular antigen of interest (or an mRNA or mRNAs encoding antigens of interest). In exemplary aspects, the vaccines of the invention feature an mRNA or mRNAs encoding antigen(s) derived from infectious diseases or cancers.

Diseases or conditions, in some embodiments include those caused by or associated with infectious agents, such as bacteria, viruses, fungi and parasites. Non-limiting examples of such infectious agents include Gram-negative bacteria, Gram-positive bacteria, RNA viruses (including (+)ssRNA viruses, (−)ssRNA viruses, dsRNA viruses), DNA viruses (including dsDNA viruses and ssDNA viruses), reverse transcriptase viruses (including ssRNA-RT viruses and dsDNA-RT viruses), protozoa, helminths, and ectoparasites.

Thus, the invention also encompasses infectious disease vaccines. The antigen of the infectious disease vaccine is a viral or bacterial antigen.

In some embodiments, a disease, disorder or condition is caused by or associated with a virus.

The compositions of the invention are also useful for treating or preventing a symptom of diseases characterized by missing or aberrant protein activity, by replacing the missing protein activity or overcoming the aberrant protein activity. Because of the rapid initiation of protein production following introduction of mRNAs, as compared to viral DNA vectors, the compounds of the present disclosure are particularly advantageous in treating acute diseases such as sepsis, stroke, and myocardial infarction. Moreover, the lack of transcriptional regulation of the alternative mRNAs of the present disclosure is advantageous in that accurate titration of protein production is achievable. Multiple diseases are characterized by missing (or substantially diminished such that proper protein function does not occur) protein activity. Such proteins may not be present, are present in very low quantities or are essentially non-functional. The present disclosure provides a method for treating such conditions or diseases in a subject by introducing polynucleotide or cell-based therapeutics containing the alternative polynucleotides provided herein, wherein the alternative polynucleotides encode for a protein that replaces the protein activity missing from the target cells of the subject.

Diseases characterized by dysfunctional or aberrant protein activity include, but are not limited to, cancer and other proliferative diseases, genetic diseases (e.g., cystic fibrosis), autoimmune diseases, diabetes, neurodegenerative diseases, cardiovascular diseases, and metabolic diseases. The present disclosure provides a method for treating such conditions or diseases in a subject by introducing polynucleotide or cell-based therapeutics containing the polynucleotides provided herein, wherein the polynucleotides encode for a protein that antagonizes or otherwise overcomes the aberrant protein activity present in the cell of the subject.

In some embodiments, microbial growth within a composition disclosed herein is inhibited. In some embodiments, microbial growth is inhibited by the compound (e.g., compound of Formula I, Formula II, and/or Formula III). In some embodiments, a composition disclosed herein does not comprise a pharmaceutical preservative. Non-limiting examples of pharmaceutical preservatives include methyl paragen, ethyl paraben, propyl paraben, butyl paraben, benzyl alcohol, chlorobutanol, phenol, meta cresol (m-cresol), chloro cresol, benzoic acid, sorbic acid, thiomersal, phenylmercuric nitrate, bronopol, propylene glycol, benzylkonium chloride, and benzethionium chloride. In some embodiments, a composition disclosed herein does not comprise phenol, m-cresol, or benzyl alcohol. Compositions in which microbial growth is inhibited may be useful in the preparation of injectable formulations, including those intended for dispensing from multi-dose vials. Multi-dose vials refer to containers of pharmaceutical compositions from which multiple doses can be taken repeatedly from the same container. Compositions intended for dispensing from multi-dose vials typically must meet USP requirements for antimicrobial effectiveness. In some embodiments, a composition disclosed herein comprising a compound (e.g., a compound of Formula I, Formula II, and/or Formula III) has antimicrobial effectiveness, and may be dispensed from a multi-dose vial.

In some embodiments, “administering” or “administration” means providing a material to a subject in a manner that is pharmacologically useful. In some embodiments, a composition disclosed herein is administered to a subject enterally. In some embodiments, an enteral administration of the composition is oral. In some embodiments, a composition disclosed herein is administered to the subject parenterally. In some embodiments, a composition disclosed herein is administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs.

To “treat” a disease as the term is used herein, means to reduce the frequency or severity of at least one sign or symptom of a disease, disorder or condition experienced by a subject. The compositions described above or elsewhere herein are typically administered to a subject in an effective amount, that is, an amount capable of producing a desirable result. The desirable result will depend upon the active agent being administered. For example, an effective amount of a composition comprising a nucleic acid, a lipid, and a compound of Formula I may be an amount of the composition that is capable of increasing expression of a protein in the subject. A therapeutically acceptable amount may be an amount that is capable of treating a disease or condition, e.g., a disease or condition that that can be relieved by increasing expression of a protein in a subject. As is well known in the medical and veterinary arts, dosage for any one subject depends on many factors, including the subject's size, body surface area, age, the particular composition to be administered, the active ingredient(s) in the composition, the intended outcome of the administration, time and route of administration, general health, and other drugs being administered concurrently.

In some embodiments, a subject is administered a composition comprising a nucleic acid, a lipid, and/or a compound of Formula I in an amount sufficient to increase expression of a protein in the subject.

In certain embodiments, LNP preparations (e.g., populations or formulations) are analyzed for polydispersity in size (e.g., particle diameter) and/or composition (e.g., amino lipid amount or concentration, phospholipid amount or concentration, structural lipid amount or concentration, PEG-lipid amount or concentration, mRNA amount (e.g., mass) or concentration) and, optionally, further assayed for in vitro and/or in vivo activity. Fractions or pools thereof can also be analyzed for accessible mRNA and/or purity (e.g., purity as determined by reverse-phase (RP) chromatography).

Particle size (e.g., particle diameter) can be determined by Dynamic Light Scattering (DLS). DLS measures a hydrodynamic diameter. Smaller particles diffuse more quickly, leading to faster fluctuations in the scattering intensity and shorter decay times for the autocorrelation function. Larger particles diffuse more slowly, leading to slower fluctuations in the scattering intensity and longer decay times in the autocorrelation function.

mRNA purity can be determined by reverse phase high-performance liquid chromatography (RP-HPLC) size based separation. This method can be used to assess mRNA integrity by a length-based gradient RP separation and UV detection of RNA at 260 nm. As used herein “main peak” or “main peak purity” refers to the RP-HPLC signal detected from mRNA that corresponds to the full size mRNA molecule loaded within a given LNP formulation. mRNA purity can also be assessed by fragmentation analysis. Fragmentation analysis (FA) is a method by which nucleic acid (e.g., mRNA) fragments can be analyzed by capillary electrophoresis. Fragmentation analysis involves sizing and quantifying nucleic acids (e.g., mRNA), for example by using an intercalating dye coupled with an LED light source. Such analysis may be completed, for example, with a Fragment Analyzer from Advanced Analytical Technologies, Inc.

Compositions formed via the methods described herein may be particularly useful for administering an agent to a subject in need thereof. In some embodiments, the compositions are used to deliver a pharmaceutically active agent. In some instances, the compositions are used to deliver a prophylactic agent. The compositions may be administered in any way known in the art of drug delivery, for example, orally, parenterally, intravenously, intramuscularly, subcutaneously, intradermally, transdermally, intrathecally, submucosally, sublingually, rectally, vaginally, etc.

Once the compositions have been prepared, they may be combined with pharmaceutically acceptable excipients to form a pharmaceutical composition. As would be appreciated by one of skill in this art, the excipients may be chosen based on the route of administration as described below, the agent being delivered, and the time course of delivery of the agent.

Pharmaceutical compositions described herein and for use in accordance with the embodiments described herein may include a pharmaceutically acceptable excipient. As used herein, the term “pharmaceutically acceptable excipient” means a non-toxic, inert solid, semi-solid or liquid filler, diluent, encapsulating material or formulation auxiliary of any type. Some examples of materials which can serve as pharmaceutically acceptable excipients are sugars such as lactose, glucose, and sucrose; starches such as corn starch and potato starch; cellulose and its derivatives such as sodium carboxymethyl cellulose, methylcellulose, hydroxypropylmethylcellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen free water; isotonic saline; citric acid, acetate salts, Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and/or to animals, orally, rectally, parenterally, intracisternally, intravaginally, intranasally, intraperitoneally, topically (as by powders, creams, ointments, or drops), bucally, or as an oral or nasal spray.

Liquid dosage forms for oral administration include pharmaceutically acceptable emulsions, microemulsions, solutions, suspensions, syrups, and elixirs. In addition to the active ingredients (i.e., the particles), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3 butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, ethanol, U.S.P., and isotonic sodium chloride solution. In addition, sterile, fixed oils are conventionally employed as a solvent or suspending medium. For this purpose any bland fixed oil can be employed including synthetic mono or diglycerides. In addition, fatty acids such as oleic acid are used in the preparation of injectables.

The injectable formulations can be sterilized, for example, by filtration through a bacteria retaining filter, or by incorporating sterilizing agents in the form of sterile solid compositions which can be dissolved or dispersed in sterile water or other sterile injectable medium prior to use.

Compositions for rectal or vaginal administration may be suppositories which can be prepared by mixing the particles with suitable non irritating excipients or carriers such as cocoa butter, polyethylene glycol, or a suppository wax which are solid at ambient temperature but liquid at body temperature and therefore melt in the rectum or vaginal cavity and release the particles.

Solid dosage forms for oral administration include capsules, tablets, pills, powders, and granules. In such solid dosage forms, the particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard filled gelatin capsules using such excipients as lactose or milk sugar as well as high molecular weight polyethylene glycols and the like.

The solid dosage forms of tablets, dragées, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a pharmaceutical composition include ointments, pastes, creams, lotions, gels, powders, solutions, sprays, inhalants, or patches. The particles are admixed under sterile conditions with a pharmaceutically acceptable carrier and any needed preservatives or buffers as may be required. Ophthalmic formulation, ear drops, and eye drops are also possible.

The ointments, pastes, creams, and gels may contain, in addition to the compositions of this invention, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the compositions of this invention, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compositions in a proper medium. Absorption enhancers can also be used to increase the flux of the compound across the skin. The rate can be controlled by either providing a rate controlling membrane or by dispersing the compositions in a polymer matrix or gel.

In other embodiments, the stabilized compositions of the invention are loaded and stored in prefilled syringes and cartridges for patient-friendly autoinjector and infusion pump devices.

Kits for use in preparing or administering the compositions are also provided. A kit for forming compositions may include any solvents, solutions, buffer agents, acids, bases, salts, targeting agent, etc. needed in the composition formation process. Different kits may be available for different targeting agents. In certain embodiments, the kit includes materials or reagents for purifying, sizing, and/or characterizing the resulting compositions. The kit may also include instructions on how to use the materials in the kit. The one or more agents (e.g., pharmaceutically active agent) to be contained within the composition are typically provided by the user of the kit.

Kits are also provided for using or administering the compositions. The compositions may be provided in convenient dosage units for administration to a subject. The kit may include multiple dosage units. For example, the kit may include 1-100 dosage units. In certain embodiments, the kit includes a week supply of dosage units, or a month supply of dosage units. In certain embodiments, the kit includes an even longer supply of dosage units. The kits may also include devices for administering the compositions. Exemplary devices include syringes, spoons, measuring devices, etc. The kit may optionally include instructions for administering the compositions (e.g., prescribing information).

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As disclosed herein, the terms “composition” and “formulation” are used interchangeably.

The following examples are intended to illustrate certain embodiments of the present invention, but do not exemplify the full scope of the invention. Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever.

### EXAMPLES

Accelerated and real-time stability data have been generated showing that methylene blue is a potent stabilizing excipient for mRNA-lipid nanoparticle (mRNA-LNP) compositions. Specifically, methylene blue concentrations in the range of 0.5-2 mM have been shown to dramatically inhibit the rate of purity loss of mRNA encapsulated within the LNP when exposed to 5° C., 25° C. and 40° C. storage conditions. This stabilization effect has also been shown for the closely related phenothiazinium compounds azure A, azure B, leucomethylene blue, and the azonium dye, safranin O. The stabilizing effect of methylene blue has been demonstrated for various mRNA sequences and LNP lipid compositions. The instability of mRNA, specifically loss of purity, is considered one of the greatest challenges to its fundamental therapeutic and commercial viability. Furthermore, the instability of mRNA is significantly worsened when formulated as an LNP. Extensive screening studies previously performed failed to identify excipients that improve the fragmentation instability of mRNA. Because methylene blue possesses low toxicity, has a >100-year history of clinical use, is available as a USP grade reagent, and is already FDA-approved as a therapeutic entity, it represents a rare example of an RNA-binding dye that is suitable for use as a pharmaceutical ingredient (Osterberg, et al., International Journal of Toxicology 22:377-380 (2003)). Molecular dyes are generally toxic and are not used as pharmaceutical excipients. Although there is precedent for analytical/biophysical uses of methylene blue and other phenothiazinium dyes in combination with RNA and DNA, there appears to be no precedent for RNA-lipid nanoparticle compositions with methylene blue or related compounds, nor for the use of methylene blue/related compounds as a stabilizing biopharmaceutical excipient. The discovery that methylene blue and certain related compounds stabilize mRNA within the LNP is thus unexpected and unprecedented. This discovery enables several significant applications, including extended refrigerated liquid shelf-life, extended in-use periods at room temperature, and extended in-use stability at physiological temperatures up to 40° C. Achieving a stable liquid formulation would also enable more commercially and therapeutically desirable packaging and delivery options including prefilled syringes and cartridges for patient-friendly autoinjector and infusion pump devices. The incorporation of this compound into process streams (potentially with subsequent removal) and final drug product is expected to provide a significant improvement in initial purity value upon manufacture, which is currently problematic, as a 5-10% purity loss during LNP formation and processing is typical with current large-scale LNP production. The ability to stabilize solutions and pharmaceutical preparations of mRNA using methylene blue and similar low toxicity additives could therefore represent a valuable disruptive technology and facilitate broader use of mRNA compositions.

### Example 1

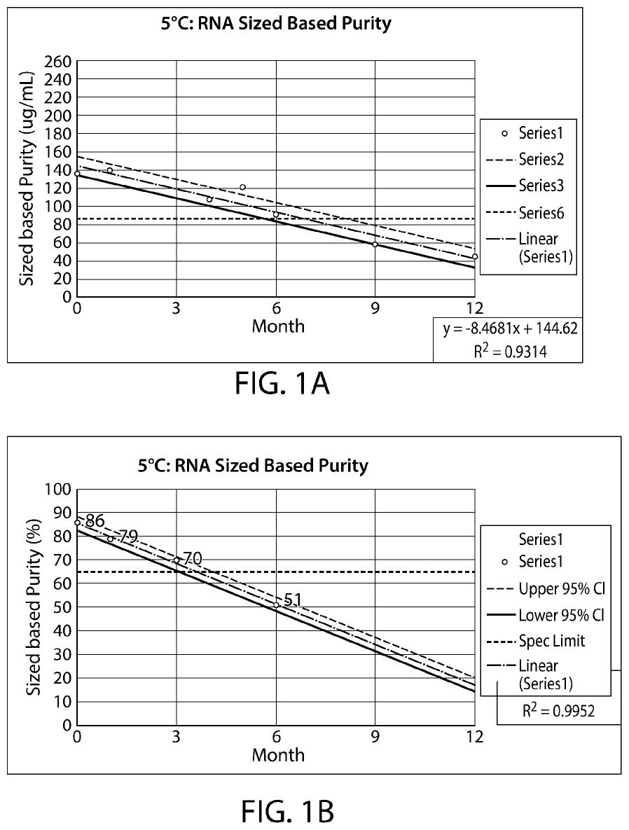

This example describes the instability of RNA in lipid nanoparticle formulations when stored as a refrigerated liquid. One of the most formidable barriers to translating the concept of using messenger RNA as a pharmaceutical agent is the inherent instability of the mRNA molecule. RNA is highly susceptible to chemical and enzymatic cleavage as well as adduct formation, which causes a loss of translational potency. Lipid nanoparticle (LNP) formulations of mRNA undergo rapid loss of purity when stored as a refrigerated liquid, as exemplified by the data in FIGS. 1A and 1B.

It is evident that the stability of mRNA is poorer when encapsulated in LNP than when stored unformulated as a simple solution in buffer. A two-year shelf-life is generally considered the minimum shelf-life target for a viable pharmaceutical product, or in the case of a poorly stable drug, 18 months at the absolute minimum. FIGS. 1A and 1B demonstrate that the shelf life of LNP-mRNA formulations falls below this minimum. Consequently, most mRNA formulations must be stored frozen at −20° C. or −80° C. Although these storage conditions may be viable in the case of rare disease treatment or highly specialized indications, they are far from ideal. Additionally, refrigerated liquid products are preferred over reconstituted lyophilized powder or −80° C. products as they are more patient-friendly for widespread use. The ability to formulate mRNA drug products in refrigerated liquid compositions would facilitate widespread use of mRNA drugs, such as for vaccine products, which are typically provided as shelf-stable injectables requiring no special reconstitution or storage conditions.

### Example 2

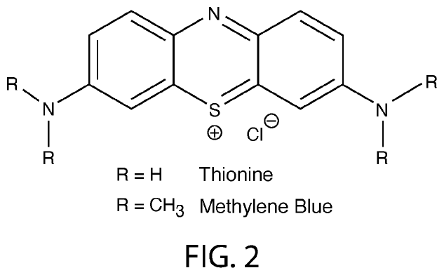

This example describes phenothiazinium dyes which interact with mRNA and their impact on LNP formulations. Prior work demonstrated that phenothiazinium dyes bind to mRNA. Certain phenothiazinium dyes such as thionine, azure A and azure B can permeate lipid nanoparticles (LNP) and bind to encapsulated mRNA without altering the gross structure of the LNP. These interpretations were made based on extensive optical spectroscopic studies, Cryo-EM, dynamic light scattering (DLS) and isothermal titration calorimetry studies. The structures of methylene blue and thionine are shown in FIG. 2. Surprisingly, it was discovered that methylene blue does not permeate LNPs to the same extent as certain other phenothiazinium dyes, even though it does bind to mRNA free in solution in an analogous manner to thionine.

Methylene blue and phenothiazine dyes are well known to cause cleavage of the phosphodiester bonds in DNA and have been widely studied as photosensitizing agents (OhUigin, et al., Nucleic Acids Research 15:7411-7427 (1987); Specht, Photochemistry and Photobiology 59:506-514 (2014); Tuite and Kelly, Journal of Photochemistry and Photobiology B: Biology 21:103-124 (1993)). This photo-reactivity of methylene blue forms the basis of its long-standing use for virus and pathogen inactivation in whole blood (Wagner, Transfusion Medicine Reviews 16:61-66 (2002); Schlenke, Transfus. Med. Hemother. 41:309-325 (2014); Wainwright, et al., Journal of Photochemistry and Photobiology B: Biology 86:45-58 (2007)). Phenothiazines have been described as “ . . . arguably the most potent chain breaking antioxidant ever identified.” (Ohlow and Moosmann, Drug Discovery Today 16:119-131 (2011)) This statement highlights the unexpectedness of the discovery disclosed herein that methylene blue (the prototypical phenothiazine) protects mRNA against degradation in the context of mRNA-LNP compositions. Literature has described methylene blue and the phenothiazinium dyes, pertaining to their use in clinical, pharmacological, photochemistry, analytical and biophysical applications among others (Oz, et al., Med. Res. Rev. 31:93-117 (2011); Ginimuge and Jyothi, J. Anaesthesiol. Clin. Pharmacol. 26:517-520 (2010); Wainwright, Biotechnic. & Histochemistry: Official Publication of the Biological Stain Commission 78:147-155 (2003); Rabinowitch and Epstein, Journal of the American Chemical Society 63:69-78 (1941); Lu, et al., BMC Medicine 16:59 (2018)). Methylene blue interacts with nucleic acids, proteins, and lipids and induces photosensitized reactions after photoactivation. It generates singlet oxygen very efficiently, causing photooxidative damage in biological systems, including strand breakage in DNA. Methylene blue can readily accept and donate electrons from and to a variety of compounds, allowing it to be either prooxidant or antioxidant under different conditions (Oz, et al., Med. Res. Rev. 31:93-117 (2011)).

Historically, the photophysical behavior of methylene blue and of its derivatives interacting with single- and double-stranded oligonucleotides has been studied extensively (Rohs, et al., Journal of the American Chemical Society 122:2860-2866 (2000)). In view of the known reactivity of phenothiazinium dyes with oligonucleotides (OhUigin, et al., Nucleic Acids Research 15:7411-7427 (1987)) it was expected that a phenothiazinium dye such as methylene blue would fail to stabilize mRNA in LNP formulations. Despite literature suggesting phenothiazinium dyes should be detrimental to nucleic acid stability, the effect of methylene blue on mRNA-LNP compositions was evaluated.

Demonstration of Methylene Blue Stabilization Effect and Comparison with Thionine

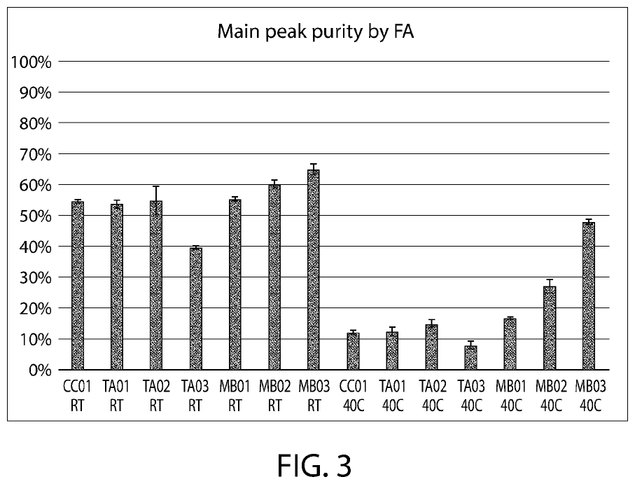

A study was performed to compare the effects of thionine, a phenothiazinium dye that permeates LNPs, with methylene blue which permeates LNPs to a lesser extent. A series of mRNA-LNP samples were prepared containing incremental levels of each dye as detailed in Table 1. These samples were incubated under two temperature conditions of room temperature and 40 C for 3 weeks prior to analysis. Stability results, measured according to main peak purity by fragmentation analysis (FA), are shown in FIG. 3. As expected, samples incubated at 40 C showed higher levels of degradation than those incubated at room temperature across all conditions. Samples containing low concentrations of thionine dye (0.01 mM and 0.1 mM) showed similar degradation as the sample with no dye at both room temperature and 40° C. However the sample containing 1.5 mM thionine showed greater degradation than the no-dye comparator. This effect occurred at both temperature conditions, suggesting that thionine is deleterious to the fragmentation stability of mRNA inside the LNP. In contrast, all samples containing methylene blue showed significantly improved mRNA stability relative to the no-dye control at both temperature conditions. Furthermore, samples containing methylene blue at both temperature conditions showed a concentration-dependency, whereby increases in methylene blue concentration were accompanied by increases in mRNA stability.

These results demonstrate that methylene blue significantly enhances the stability of mRNA encapsulated within LNPs at room temperature and at 40° C. In contrast, thionine decreased mRNA stability under these conditions. Without wishing to be bound to theory, this stabilization effect could be the result of methylene blue sequestering a reactive species of the LNP.

TABLE 1
Sample information corresponding to the data of FIG. 3.

| Sample | LNP nominal | | Dye Concentration |
|---|---|---|---|
| ID | concentration | Dye | (mM) |
| CC00 | 0.5 | None | 0 |
| TA01 | 0.5 | Thionine | 0.01 |
| TA02 | 0.5 | Thionine | 0.1 |
| TA03 | 0.5 | Thionine | 1.5 |
| MB01 | 0.5 | Methylene Blue | 0.01 |
| MB02 | 0.5 | Methylene Blue | 0.1 |
| MB03 | 0.5 | Methylene Blue | 1.5 |

### Example 3

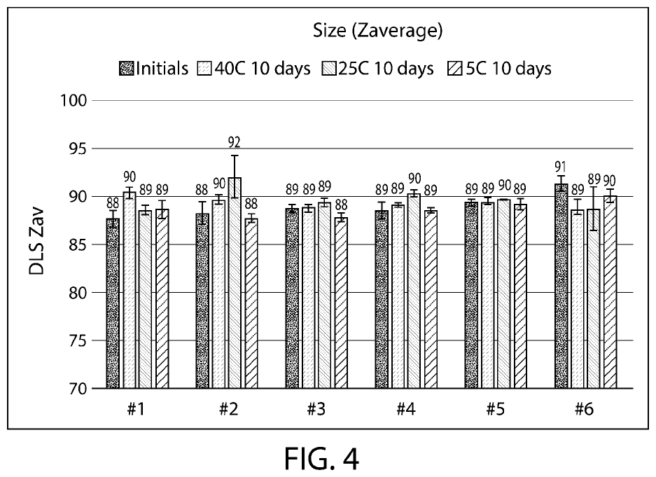

In view of the surprising and somewhat counter-intuitive finding that methylene blue enhanced stability of mRNA-LNP formulations, additional experimentation was conducted. The stability of mRNA-LNPs in the presence of increasing levels of methylene blue was investigated upon incubation at 5° C., 25° C. or 40° C. for 10 days. The methylene blue concentrations for each experimental condition are shown in Table 2. Initial result showed that 3 weeks at 40° C. produced near complete mRNA degradation in the absence of methylene blue, so the experiment was not extended for longer periods of time. In addition to chemical stability, a careful examination of the possible impact of methylene blue on the physical state of the LNP was performed using dynamic light scattering (DLS). This technique is exquisitely sensitive to tiny changes in hydrodynamic nanoparticle size, especially with respect to the formation of larger aggregates known to be a common physical degradation mode of many LNPs. The presence of up to 6 mM methylene blue had no impact on the physical stability of the mRNA-LNP under the three temperature conditions tested (FIG. 4).

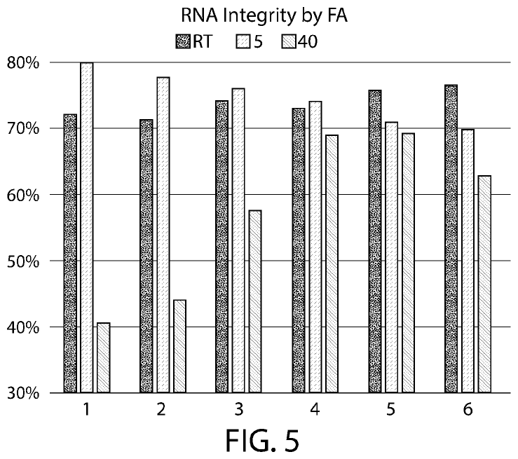

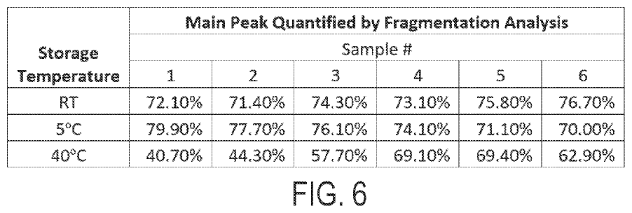

Evaluating RNA integrity by fragmentation analysis further demonstrated that methylene blue enhances RNA stability in LNP compositions (FIG. 5 and FIG. 6). Increasing levels of methylene blue demonstrated a significant stabilizing effect on mRNA within LNP compositions. The 40° C. data suggest that this effect reached a maximum in the range of ˜1.5-3.0 mM methylene blue. The room temperature data also show a protective effect of the dye. However, the 5° C. results show a slight trend in the opposite direction, though overall mRNA stability in the LNP-methylene blue compositions remained high.

TABLE 2
Sample designations used in Example 3
(FIG. 4, FIG. 5 and FIG. 6), and their corresponding
methylene blue (MB) concentrations.

| Sample # | [MB], mM |
|---|---|
| 1 | 0 |
| 2 | 0.1 |
| 3 | 0.5 |
| 4 | 1.5 |
| 5 | 3 |
| 6 | 6 |

### Example 4

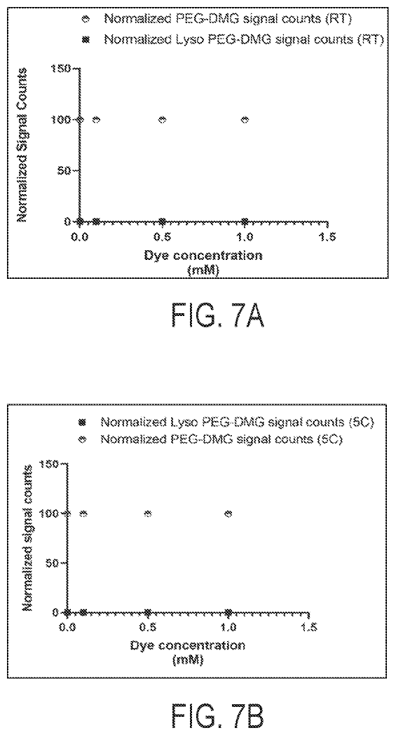

This example describes the effects of methylene blue on lipid components of LNP formulations. PEG-lipid is known to be susceptible to oxidative degradation. Given the degradation mechanism attributed to methylene blue, an accelerated study was performed to evaluate whether the presence of methylene blue adversely affects PEG-lipid stability. Lyso-PEG-DMG is considered a primary bellwether of PEG-lipid degradation. As such, its concentration in samples was measured using liquid chromatography/mass spectrometry (LC/MS) after storage of LNPs at room temperature (FIG. 7A) or 5° C. (FIG. 7B) in the presence of a range of methylene blue concentrations. Over the range of 0 to 1.0 mM methylene blue, no difference in Lyso-PEG-DMG signal was detected, suggesting that methylene blue has no adverse effects on the PEG-lipid of LNP formulations.

### Example 5

This example describes experimentation demonstrating additional evidence of the efficacy of methylene blue to stabilize LNP formulations of mRNA. In this example, a formulation containing two distinct mRNA sequences in the LNP formulation was used.

### Study Design

Given the results demonstrated above, additional experimentation was designed to extend the previous results to further mRNA-LNP formulations; to include additional controls to determine whether the presence of the dye itself influences analytical results; to evaluate the effects of light exposure, given the photodependence of phenothiazinium dye reactivity; to evaluate longer-term stability of methylene blue-containing refrigerated formulations; and to expand the method of evaluating mRNA purity to a second modality, i.e. reverse phase HPLC (RP-HPLC) in addition to fragmentation analysis.

As detailed in this example, samples are labeled as follows: A=0 mM methylene blue; B=0.5 mM methylene blue; C=1 mM methylene blue; D=1.5 mM methylene blue; and E=2.0 mM methylene blue.

### Effects of Methylene Blue on LNP Physical Stability

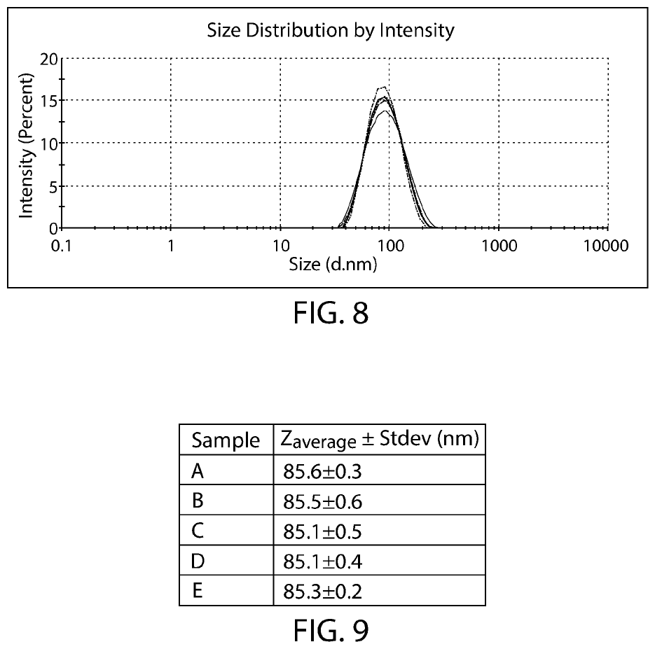

To confirm that methylene blue does not affect the physical stability of the particles themselves within LNP formulations, samples mRNA-LNP samples were incubated with 0-2 mM methylene blue in refrigerated storage for 3 weeks. After the 3 week incubation, samples were analyzed by dynamic light scattering (DLS) to characterize the hydrodynamic diameters of LNPs. Each of the samples (e.g., LNP compositions with 0, 0.5, 1.0, 1.5 and 2.0 mM methylene blue) demonstrated the same LNP size distributions and average hydrodynamic diameters after 3 weeks refrigerated storage (FIG. 8 and FIG. 9), demonstrating that methylene blue does not have an impact on the overall lipid nanoparticle structure.

Effect of 25° C., 40° C. and Light on mRNA Stability

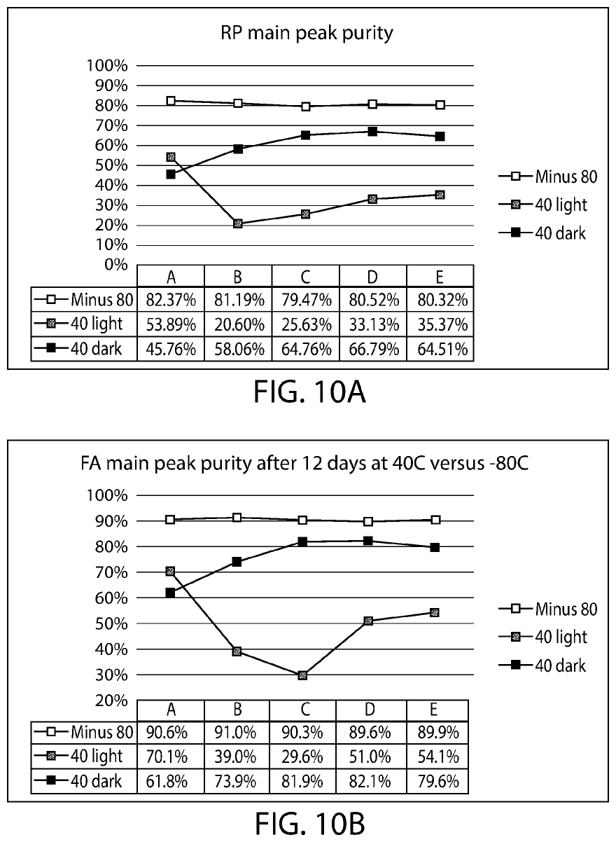

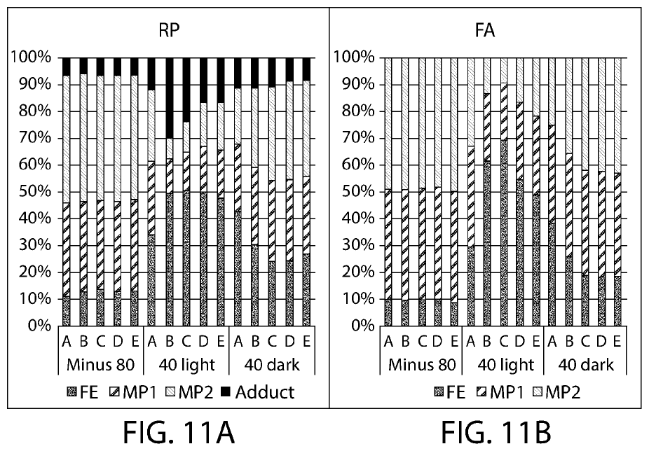

After incubating samples at 40° C. for 12 days, either protected from light or unprotected, fragmentation analysis was conducted to evaluate the presence of lipid-RNA adducts. Comparison of the samples incubated at 40° C. for 12 days with control samples that had been stored at −80 C demonstrated that increasing methylene blue concentrations enhanced the stability of mRNA in LNP formulations, as measured by RP-HPLC (FIG. 10A and FIG. 11A) and by fragmentation analysis (FIG. 10B and FIG. 11B). These results additionally demonstrated that samples containing methylene blue (i.e., samples B-E) and stored unprotected from light had more mRNA degradation than samples stored protected from light or those without methylene blue.

### Major Interpretations

The results of this example demonstrate that methylene blue stabilizes LNP with respect to mRNA fragmentation under accelerated temperature conditions with samples protected from light. The data from the −80° C. samples in FIG. 10A and FIG. 10B show that the presence of methylene blue did not interfere with measurement methods used herein. Samples stored at 40° C. and exposed to light showed greater degradation with respect to both fragmentation and adduct formation relative to those stored protected from light. Methylene blue was previously shown to be photoactivated to become a potent strand scission agent (Ohlow and Moosmann, Drug Discovery Today 16:119-131 (2011)), supporting the hypothesis that when protected from light, methylene blue exhibits a surprisingly different reactivity towards mRNA in LNP formulations than towards nucleic acids in other formulations when exposed to light. Furthermore, samples incubated at 40° C. and protected from light demonstrated that methylene blue did not have a detrimental effect on adduct formation, and may in fact have improved stability to some extent, although this stabilization effect may not be statistically significant in this particular dataset.

These data show that the stabilizing effect of methylene blue applies generally across mRNA sequences, seeing as in this example, a mRNA-LNP formulation was used containing two mRNA sequences. Additionally, the presence of up to 2 mM methylene blue did not impact the physical stability of LNP formulations stored refrigerated as shown by DLS measurements after 3 weeks.

### Example 6

This example relates to stabilization of mRNA in LNP formulations by the addition of methylene blue over long-term refrigerated storage. The preceding examples with methylene blue demonstrated a stabilization effect with respect to accelerated temperature stress conditions (i.e., 25° C. and 40° C.). To confirm that this stabilization effect would also occur with long-term refrigerated storage, a study was conducted to evaluate the effect of various concentrations of methylene blue (0, 0.5, 1, 1.5 and 2.0 mM) on the stability of a lipid nanoparticle formulation containing two distinct mRNA compounds stored at 5° C.

Physical Stability after 5 Months Refrigerated Storage

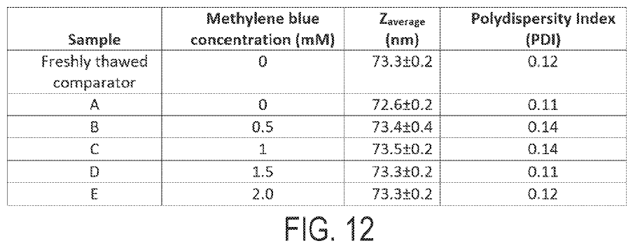

Measuring hydrodynamic diameter of LNPs after refrigerated storage for 5 months demonstrated no significant impact of methylene blue up to a concentration of 2.0 mM on the hydrodynamic size or size distribution (polydispersity index) of LNP. Furthermore, analysis of a sample of LNP from a freshly thawed vial (i.e., one stored at −80° C. and thawed for measurement) demonstrated such LNPs had equivalent size characteristics to those stored at 5° C., indicating that the LNP possesses good physical stability when stored as a refrigerated liquid in the presence or absence of methylene blue (FIG. 12). Samples were measured by dynamic light scattering (DLS) in PBS after being filtered through 0.8 μm filters to remove large particulates.

Chemical Stability after 5 Months Refrigerated Storage

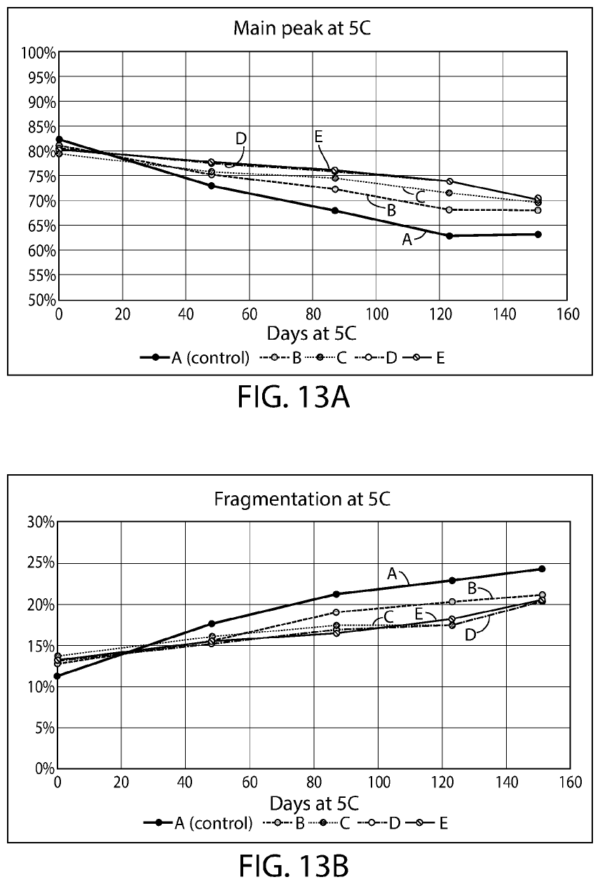

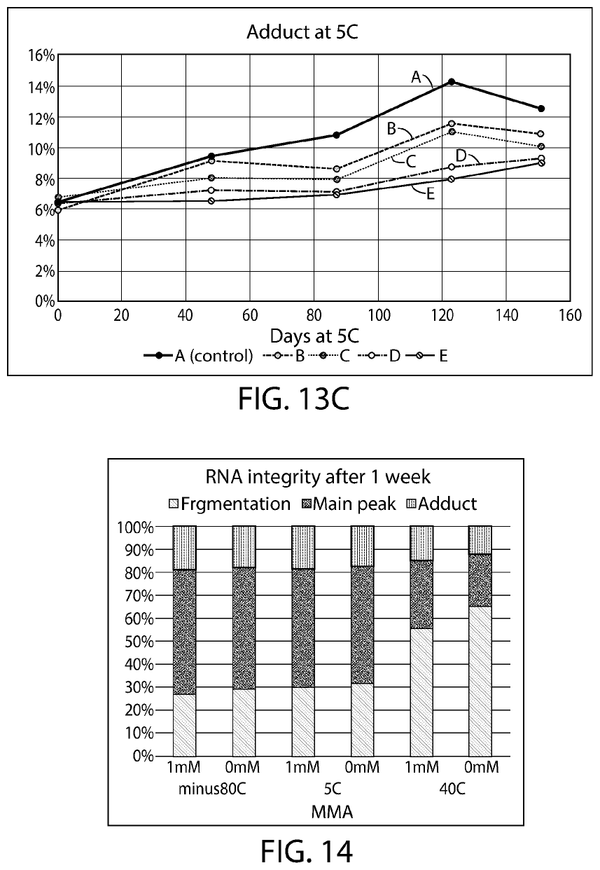

To determine the effects of methylene blue on the chemical stability of mRNA-LNP formulations over long-term refrigerated storage, mRNA purity, mRNA fragmentation, and mRNA-lipid adduct formation were measured over time in mRNA-LNP samples containing varying concentrations of methylene blue (A=0 mM, B=0.5 mM, C=1.0 mM, D=1.5 mM, E=2.0 mM methylene blue). Total purity was determined according to main peak percentage by RP-HPLC (FIG. 13A), fragmentation was determined according to fragmentation analysis (FIG. 13B), and mRNA-lipid adduct formation was determined according to RP-HPLC (FIG. 13C). With concentrations as low as 0.5 mM methylene blue, fragmentation rate was significantly inhibited relative to control, and there was little difference in the effect of 0.5 mM versus 2.0 mM. The results suggest a methylene blue concentration of 1.5 mM is optimal with respect to adduct formation.

Methylene Blue Stabilization of Lyophilized mRNA-LNP

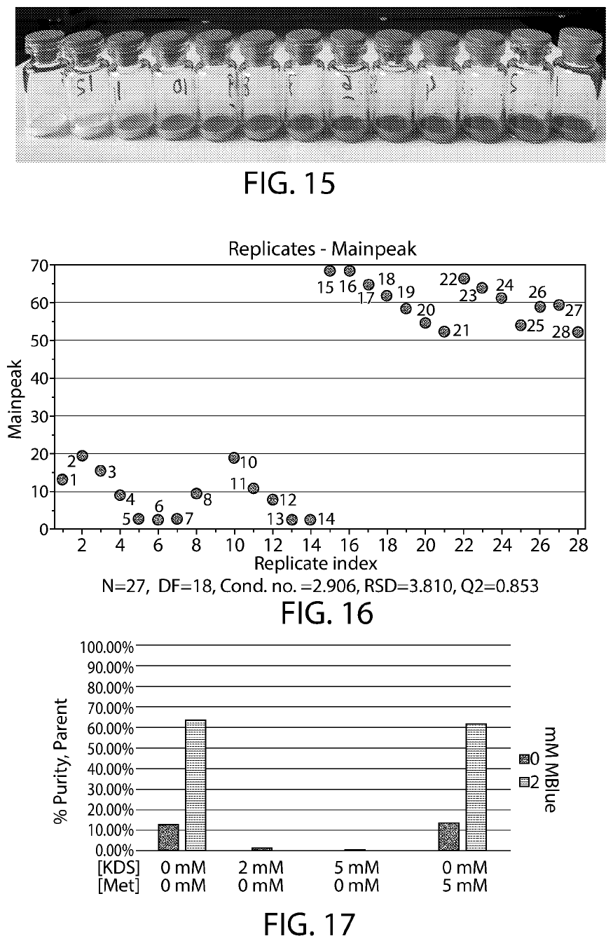

To evaluate whether methylene blue could have a stabilization effect on lyophilized powder formulations of mRNA-LNP, studies were performed to compare the effect of 1 mM methylene blue on loss of purity of a lyophilized mRNA-LNP. In this experiment methylene blue was added to LNP immediately prior to lyophilization. The resulting powders (shown in FIG. 15) were stored at −80° C., 5° C. or 40° C. with or without methylene blue. Results demonstrated that samples containing methylene blue had improved mRNA purity (33% versus 22% main peak purity after 1 week at 40° C.) as lyophilized powder in vials (FIG. 14).

### Example 7

### Methylene Blue Stabilizes Solutions of Various Compositions

To broadly evaluate the effect of methylene blue under a large number of systematically varied solution conditions, a design of experiment (DOE) approach was utilized. The study design varied pH, buffer species, NaCl and methylene blue concentration as given in Table 3.

TABLE 3
Compositions evaluated in DOE study of solution
composition in the presence and absence of methylene blue.
In the Buffer column, T and P correspond to Tris and Phosphate
buffers, respectively, and the numeral indicates
the room temperature pH value of the buffer.

| Sample # | Name | Buffer | [NaCl], mM | [MB], mM |
|---|---|---|---|---|
| 1 | N1 | T7.0 | 0 | 0 |
| 2 | N2 | T7.5 | 0 | 0 |
| 3 | N3 | T8.0 | 0 | 0 |
| 4 | N4 | T8.5 | 0 | 0 |
| 5 | N5 | P7.0 | 0 | 0 |
| 6 | N6 | P7.5 | 0 | 0 |
| 7 | N7 | P8.0 | 0 | 0 |
| 8 | N8 | T7.0 | 150 | 0 |
| 9 | N9 | T7.5 | 150 | 0 |
| 10 | N10 | T8.0 | 150 | 0 |
| 11 | N11 | T8.5 | 150 | 0 |
| 12 | N12 | P7.0 | 150 | 0 |
| 13 | N13 | P7.5 | 150 | 0 |
| 14 | N14 | P8.0 | 150 | 0 |
| 15 | N15 | T7.0 | 0 | 2 |
| 16 | N16 | T7.5 | 0 | 2 |
| 17 | N17 | T8.0 | 0 | 2 |
| 18 | N18 | T8.5 | 0 | 2 |
| 19 | N19 | P7.0 | 0 | 2 |
| 20 | N20 | P7.5 | 0 | 2 |
| 21 | N21 | P8.0 | 0 | 2 |
| 22 | N22 | T7.0 | 150 | 2 |
| 23 | N23 | T7.5 | 150 | 2 |
| 24 | N24 | T8.0 | 150 | 2 |
| 25 | N25 | T8.5 | 150 | 2 |
| 26 | N26 | P7.0 | 150 | 2 |
| 27 | N27 | P7.5 | 150 | 2 |
| 28 | N28 | P8.0 | 150 | 2 |

The results show two distinct groupings: samples without methylene blue (labeled 1-14, as described in Table 3) show main peak mRNA purity values in the range of 0-20%, whereas samples containing 2 mM methylene blue (labeled 15-28, as described in Table 3) show purity values in the range of 50-70% (FIG. 16). These data demonstrate the potent stabilizing effect of methylene blue and furthermore that the stabilization effect of this dye is effective across a wide variety of pharmaceutically relevant buffer compositions and pH values. In this study, formulations comprising 20 mM Tris buffer at pH 7.0 and 7.5 without NaCl and with 2 mM methylene blue present were the most stable (samples 15 and 16). The 14 samples formulated without methylene blue had an average purity of 9±6% whereas the 14 samples formulated with 2 mM methylene blue had an average purity of 60±6%. The strong statistical significance of the effect of 2 mM methylene blue on stability demonstrated the effect of the presence of methylene blue was far greater than the effect of any other factor investigated in this example on mRNA stability.

### Example 8

This example discusses the effects of conventional pharmaceutical antioxidants on mRNA stability. To evaluate the effects conventional pharmaceutical antioxidants might have on mRNA stability relative to methylene blue, a direct comparative study was performed between methionine, potassium metabisulfite (KDS) and methylene blue. mRNA purity was measured after incubation of mRNA-LNP compositions with no additive, or with 2 mM KDS, 5 mM KDS, or 5 mM methionine, in the presence or absence of 2 mM methylene blue. Neither KDS nor methionine showed any stabilization effect (FIG. 17), suggesting that methylene blue increases mRNA stability via a non-obvious reactivity.

### Example 9

This example discusses the effects of various dye molecules on mRNA stability within LNP formulations. To evaluate the potential stabilization effects of dyes, LNP compositions were prepared containing 2 mM of one of 25 dyes. After incubation for 4 days at 40° C., main peak mRNA purity was measured by RP-HPLC. Variance within this study was estimated from three independent sample replicates with no dye (Sample IDs D0a, D0b and D0c as shown in Table 4) or with 2 mM methylene blue (Sample IDs D1a, D1b and D1c as shown in Table 4). Based on these results, a difference of ±4% main peak mRNA purity relative to the no-dye control average was interpreted as significant. The main peak purity measured in each mRNA-LNP formulation, as well as its difference relative to the no dye controls is shown in Table 4. Positive differences relative to no dye represent improvements in mRNA stability, and negative differences represent worsened mRNA stability. Samples shown with no value in the Main Peak Purity column were physically unstable and therefore, did not yield reliable chromatographic results, and therefore the corresponding dyes were interpreted to be destabilizing to mRNA in LNP formulations. Several compounds were identified to confer stabilization effects similar to that of methylene blue, including Azure A, Azure B, Safranin O and Leucomethylene Blue (mesylate).

TABLE 4
Effects of various molecular dyes on mRNA
stability within LNP formulations.

| | | Main Peak | | |
|---|---|---|---|---|
| | | Purity | | Difference |
| Sample | | after 4 days at | | relative to |
| ID | Dye | 40° C. (%) | Mean | No Dye |
| D0a | None | 75.04 | 75 ± 2 | 0 |
| D0b | None | 72.68 | | |
| D0c | None | 76.44 | | |
| D1a | Methylene blue | 89.62 | 89 ± 2 | 14 |
| D1b | Methylene blue | 90.27 | | |
| D1c | Methylene blue | 85.96 | | |
| D2 | Thionine acetate | — | N/A | N/A |
| D3 | Azure A chloride | 90.36 | N/A | 15 |
| D4 | Azure B | 90.16 | N/A | 15 |
| D5 | Toluidine Blue O | 74.66 | N/A | 0 |
| D6 | Safranin O | 89.14 | N/A | 14 |
| D7 | New methylene blue N | 68.61 | N/A | −6 |
| D8 | Silver nitrate (0.1 N) | 77.90 | N/A | 3 |
| D9 | Acridine Orange | 69.92 | N/A | −5 |
| | hydrochloride hydrate | | | |
| D10 | Proflavine hemisulfate | — | N/A | N/A |
| | salt hydrate | | | |
| D11 | Acriflavine hydrochloride | — | N/A | N/A |
| D12 | 1,9-Dimethyl-Methylene | — | N/A | N/A |
| | Blue zinc | | | |
| | chloride double salt | | | |
| D13 | Nile Blue A | 70.75 | N/A | −4 |
| D14 | Nile Red | 77.56 | N/A | 3 |
| D15 | Bromophenol Blue | 35.53 | N/A | −39 |
| | sodium salt | | | |
| D16 | Brilliant Blue G | 37.42 | N/A | −38 |
| D17 | Hematoxylin | 39.82 | N/A | −35 |
| D18 | Neutral Red | 59.34 | N/A | −16 |
| D19 | Crystal Violet | 24.79 | N/A | −50 |
| D20 | Phenol Red | 46.76 | N/A | −28 |
| D21 | Eosin B | 78.00 | N/A | 3 |
| D22 | Carmine | 74.27 | N/A | −1 |
| D23 | Fluorescein sodium salt | 76.41 | N/A | 1 |
| D24 | Methylene green zinc | — | N/A | N/A |
| | chloride double salt | | | |
| D25 | Pyronin Y | 68.68 | N/A | −6 |
| D26 | Leucomethylene Blue | 89.46 | N/A | 14 |
| | (mesylate) | | | |

### Example 10

This example discusses stabilization effects of methylene blue on mRNA-LNP formulations in varying buffer conditions. To match experiments conducted on lyophilized products, which were reconstituted in saline, and refrigerated/frozen formulations in TRIS-Sucrose, two buffers across the range of pH were tested. Sodium chloride was added to mimic saline, as it can cause methylene blue to crash out of solution.

Table 5 summarizes the results obtained from fragmentation analysis and RP-HPLC. The data demonstrate that phosphate buffer did not improve the fragmentation of mRNA compared to TRIS sucrose. Results obtained from samples in both buffer conditions exhibited a similar pH trend, demonstrating that higher pH resulted in more fragmentation of mRNA. The concentration of NaCl was not sufficient to crash methylene blue out of solution, and it was found to have little to no effect on mRNA stability at such concentration.

TABLE 5
Results from mRNA stability experiments conducted with
mRNA-LNP formulations in differing buffer conditions
with or without methylene blue.

| | | NaCl | MB | | Main | |
|---|---|---|---|---|---|---|
| pH | Buffer | (mM) | Dye (mM) | Fragmentation | peak | Adduct |
| 7 | Phos | 150 | 0 | 17.9% | 7.9% | 74.2% |
| 7 | Phos | 150 | 2 | 17.1% | 59.1% | 23.9% |
| 7.5 | Phos | 150 | 0 | 11.7% | 2.4% | 85.9% |
| 7.5 | Phos | 150 | 2 | 18.7% | 59.4% | 22.0% |
| 8 | Phos | 150 | 0 | 14.2% | 2.4% | 83.3% |
| 8 | Phos | 150 | 2 | 25.0% | 52.2% | 22.7% |
| 7 | T-S | 150 | 0 | 22.6% | 9.4% | 68.0% |
| 7 | T-S | 150 | 2 | 16.0% | 66.4% | 17.6% |
| 7.5 | T-S | 150 | 2 | 19.5% | 64.1% | 16.4% |
| 7.5 | T-S | 150 | 0 | No data available | | |
| 8 | T-S | 150 | 0 | 57.5% | 18.9% | 23.6% |
| 8 | T-S | 150 | 2 | 22.9% | 61.2% | 15.9% |
| 8.5 | T-S | 150 | 0 | 73.7% | 10.9% | 15.5% |
| 8.5 | T-S | 150 | 2 | 32.5% | 54.1% | 13.3% |

In the Buffer column, “Phos” refers to phosphate buffer, and “T-S” refers to TRIS-sucrose buffer.

### Example 11

This example describes the effects of methylene blue on the conformational state of mRNA within mRNA-LNPs.

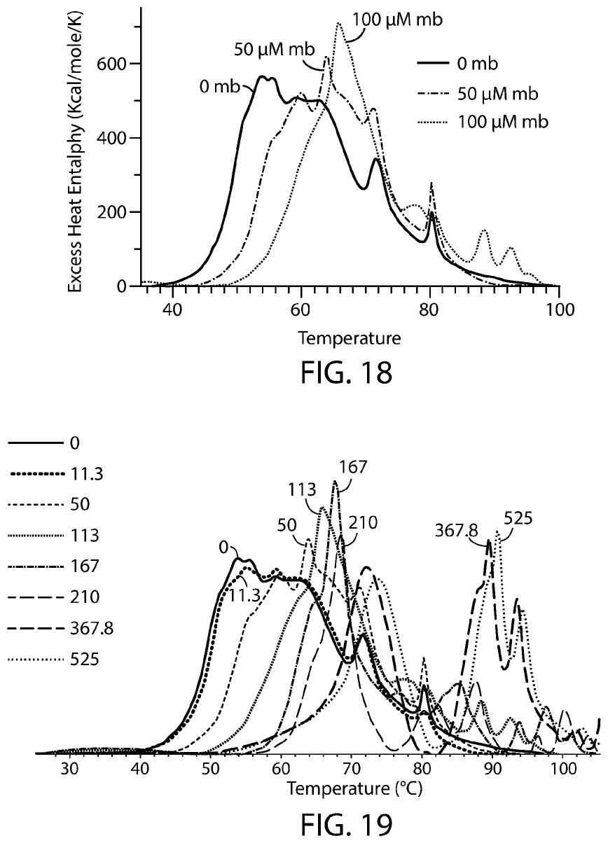

To test the effect of methylene blue on folding and intramolecular hybridization of mRNA, differential scanning calorimetry (DSC) was conducted on samples of mRNA containing no methylene blue, 50 μM methylene blue, or 100 μM methylene blue. The results shown in FIG. 18 demonstrate that there is an enormous enhancement of folded stability in the presence of small concentrations of methylene blue. At higher methylene blue concentrations (approximately 0.5 mM), precipitation of mRNA occurs, but at the concentrations of this experiment the mRNA-dye complex remains soluble. It is notable that the sharp transitions at ˜74° C. and 80° C. are not affected by the presence of dye, whereas there is a massive effect on the least thermally stable transitions occurring in the <60° C. region, shown by the large rightward shift of the curves with the addition of methylene blue. By comparison, therapeutic protein stabilization strategies commonly involve screening of excipient effects on thermal unfolding, however, the thermal shifts observed for optimized protein formulations typically involve thermal shifts of only a few degrees.

Additional DSC thermograms demonstrate the effect of incremental μM concentrations of methylene blue on mRNA structure. FIG. 19 demonstrates that concentrations of methylene blue as low as 11.3 μM affect the folded structure of mRNA at a concentration of 0.3 μM, and that incremental increases in methylene blue concentration result in incremental changes in the mRNA folded structure. The thermograms additionally demonstrate that at concentrations of methylene blue of 367.8 μM and above result in dramatic global changes in the overall mRNA structure.

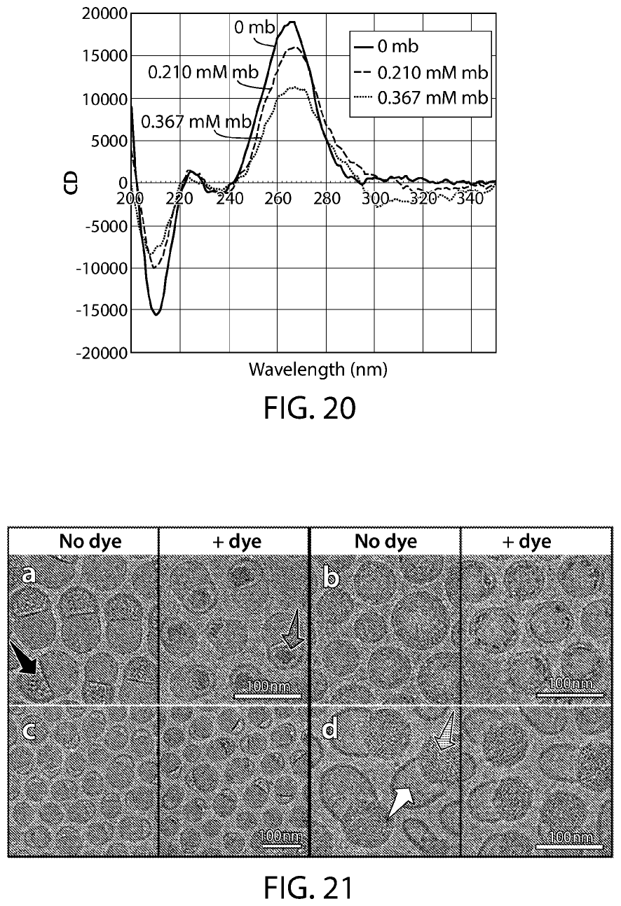

The effect of methylene blue on the conformational state of mRNA is also apparent from the circular dichroism (CD) spectra shown in FIG. 20. The CD bands correspond to the intrinsic CD of the mRNA itself. The addition of methylene blue causes a significant perturbation in these bands, demonstrating that even at very low concentrations methylene blue binding results in a major alteration to mRNA structure. The stabilizing effect of methylene blue observed on mRNA may be attributed to this binding and conformational stabilization evident from the DSC thermograms (FIGS. 18 and 19). These observations are all in accordance with the μm dissociation constant characterized by ITC for the dye-mRNA interaction.

Cryo-electron microscopy (FIG. 21) demonstrates the effects of thionine on mRNA in mRNA-LNPs of different morphologies. The images demonstrate that lipid-dissociated mRNA may reside in bleb compartments (top left images, labeled “a”) or may be more lipid associated in spherical (top right images, labeled “b”) or less prominently blebbed particles (bottom left images, labeled “c”). The arrow in the left (“No dye”) panel of the “a” images indicates the distinctive mottled mass density of mRNA inside the bleb cavity which itself is distinguished by a thick, dark periphery. The arrow in the right (“+ dye”) panel of the “a” images indicates the significant contrast enhancement that occurs when thionine dye is present, thereby identifying mRNA within the bleb. The bottom right images, labeled “d” show charge-driven migration of mRNA from the bleb into the body of the LNP. When the mRNA-LNP sample of the “a” no dye panel was dialyzed into pH 5 buffer, the images of “d” resulted. The mottled density in the “No dye” panel of the “d” images is associated with the body of the LNP (top right arrow) leaving the bleb cavity devoid of mRNA (bottom left arrow). Addition of thionine to this sample produced no notable contrast change (“+ dye” panel), indicating that thionine did not displace lipid. Collectively, these results establish that dye can permeate the LNP to bind to encapsulated mRNA without disrupting the overall structural character of the LNP.

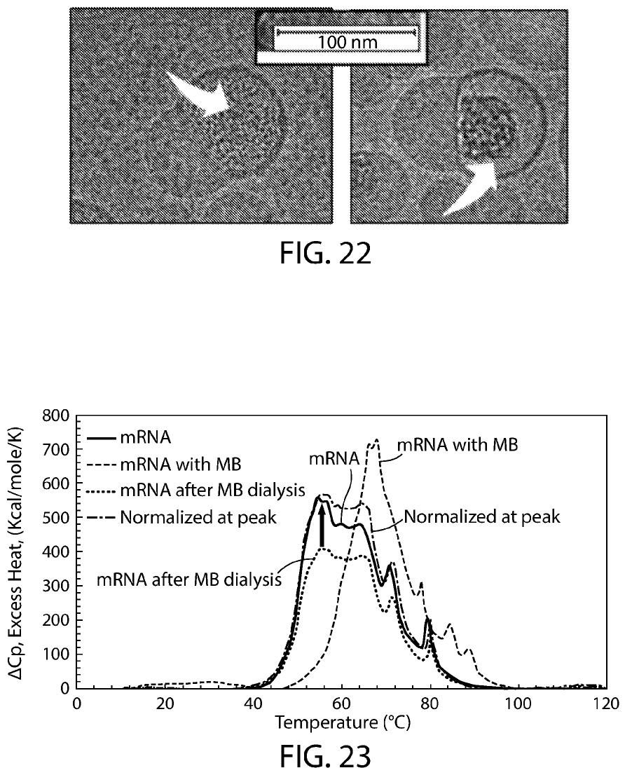

The images in FIG. 22 show the effect of thionine on mRNA-LNPs. In the left image, mRNA in the absence of dye fills the bleb, whereas the binding of dye has clearly compacted the mRNA in the right image. The significant protection against fragmentation and adduct formation conferred by the compounds disclosed herein may be based on this effect on nucleic acid conformational state. Similarly, DLS studies of the interaction between methylene blue and mRNA showed a measurable decrease in mRNA hydrodynamic radii at concentrations in the 10 μM range (data not shown).

The data in FIG. 23 demonstrate that methylene blue binding to mRNA is reversible. The DSC thermogram show the effects on the thermal unfolding profile of mRNA in the presence or absence of 100 mM methylene blue. When the methylene blue-containing sample was dialyzed to remove dye, the resulting thermogram corresponded closely to that of the original no-dye sample. This result is extremely significant because it demonstrates reversibility between the methylene blue-bound and -unbound states of mRNA. All the fine details of the thermal unfolding profile were restored post-dialysis, indicating a restoration of the original structure-function of the mRNA.

Multiple types of biophysical data presented herein (e.g., CD, ITC, and visible absorption) suggest an interaction between dye and mRNA occurring via intercalation into the mRNA double stranded regions under conditions of low dye:nucleotide ratio, i.e. conditions of excess RNA, whereas at higher dye:RNA ratios the binding mode changes to outside binding with concomitant major changes in mRNA conformation. While the precise stoichiometries applicable to pharmaceutical conditions remain to be determined, notably the DSC data shows that even in the presence of methylene blue concentrations as low as ˜10 μM, there is an appreciable impact to mRNA conformational stability (FIG. 19). This increase in conformational stability with a concomitant compaction and rigidity of the structure may form the basis for enhanced chemical stability with respect to fragmentation and adduct formation. Because the structure is rigid, labile regions of the sequence may become less accessible, thus causing it to be less energetically favorable for the mRNA molecule to adopt conformations required for degradation reactions to proceed.

The preceding examples have demonstrated that methylene blue and several other dye molecules potently confer stability on mRNA in LNP formulations. Two analytical methods, fragment analysis and RP-HPLC, were used to evaluate mRNA purity. DLS studies further showed that methylene blue did not impact the physical stability or the physical state of the LNP.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A stabilized pharmaceutical composition comprising:
a nucleic acid formulation comprising a nucleic acid and a lipid, and a compound of Formula I: 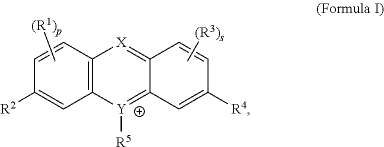
or an acceptable salt, tautomer, reduced form, or oxidized form thereof,
mixed with the nucleic acid, wherein:
Y is N, S, or O;
X is N—R⁵, S, O, or C—R^{C};
R² and R⁴ are each independently —N(R^{N})₂;
each R⁵ is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or is absent;
each instance of R¹ and R³ is independently halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, optionally substituted sulfinyl, optionally substituted sulfonyl, —OR^{O}, —N(R^{N})₂, or —SR^{S};
p is 0, 1, 2, or 3;
s is 0, 1, 2, or 3;
each instance of R^{O} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or an oxygen protecting group;
each instance of R^{N} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or a nitrogen protecting group; or two R^{N} bonded to the name nitrogen atom are taken together with the intervening atoms to form optionally substituted heterocyclyl or optionally substituted heteroaryl;
each instance of R^{S} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or a nitrogen protecting group; and
R^{C} is hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, optionally substituted sulfinyl, optionally substituted sulfonyl, —OR^{O}, —N(R^{N})₂, or —SR^{S}.

2. The composition of claim 1, wherein said nucleic acid formulation comprises lipid nanoparticles.

3. The composition of claim 2, wherein said nucleic acid formulation comprises liposomes.

4. The composition of claim 2, wherein said nucleic acid formulation comprises a lipoplex.

5. The composition of any one of claims 2 to 4, wherein the nucleic acid is encapsulated within the lipid nanoparticles, liposomes, or lipoplex.

6. The composition of any one of claims 1 to 5, wherein the nucleic acid is mRNA.

7. The composition of any preceding claim, wherein the compound is a compound of Formula II: 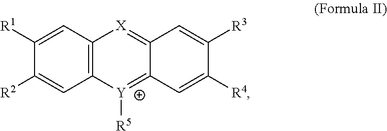
or an acceptable salt, tautomer, reduced form, or oxidized form thereof.

8. The composition of any preceding claim, wherein the compound is a compound of Formula III: 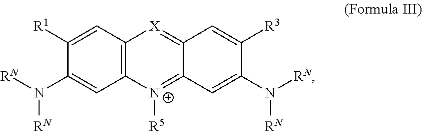
or an acceptable salt, tautomer, reduced form, or oxidized form thereof.

9. The composition of any preceding claim, wherein the compound is not thionine or a salt thereof.

10. The composition of any preceding claim, wherein the compound is methylene blue, acriflavine, toluidine blue O, safranin O, phenosafranin, leucomethylene blue or any mixture thereof.

11. The composition of any preceding claim, wherein the compound is methylene blue, acriflavine, safranin O, phenosafranin, leucomethylene blue or any mixture thereof.

12. The composition of any preceding claim, wherein the compound is methylene blue.

13. The composition of any preceding claim, wherein the compound has a purity of at least 70%, 80%, 90%, 95%, or 99%.

14. The composition of any preceding claim, wherein the compound contains fewer than 100 ppm of elemental metals.

15. The composition of any preceding claim, wherein the composition is formulated in an aqueous solution.

16. The composition of claim 15, wherein the aqueous solution comprises lipid nanoparticles and wherein the nucleic acid is encapsulated in the lipid nanoparticles.

17. The composition of claim 15 or 16, wherein the aqueous solution has a pH of or about 5 to 8, including pH of about 5, 5.5, 6, 6.5, 7, 7.5, or 8.

18. The composition of claim 15 or 16, wherein the aqueous solution does not comprise NaCl.

19. The composition of claim 15 or 16, wherein the aqueous solution comprises NaCl in a concentration of or about 150 mM.

20. The composition of any one of claims 15-19, wherein the aqueous solution comprises a phosphate buffer, a tris buffer, an acetate buffer, a histidine buffer, or a citrate buffer.

21. The composition of any one of claims 15-20, wherein the compound is present at a concentration between about 0.1 mM and about 3 mM.

22. The composition of any one of claims 15-21, wherein the compound is present at a concentration of or about 2 mM.

23. The composition of any one of claims 15-21, wherein the compound is present at a concentration of or about 1 mM.

24. The composition of any one of claims 15-21, wherein the compound is present at a concentration of or about 0.5 mM.

25. The composition of any one of claims 1 to 17, wherein the nucleic acid is a lyophilized product.

26. The composition of claim 25, wherein the lyophilized product comprises lipid nanoparticles wherein the nucleic acid is encapsulated in the lipid nanoparticles.

27. A stabilized pharmaceutical composition comprising:
a nucleic acid formulation comprising a nucleic acid and a lipid, and methylene blue, having the formula: 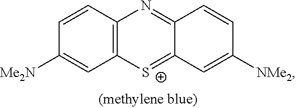 [SMILES: CN(C)c1ccc2nc3ccc(N(C)C)cc3[s+]c2c1]
mixed with the nucleic acid formulation.

28. The composition of claim 27, wherein said nucleic acid formulation comprises lipid nanoparticles.

29. The composition of claim 28, wherein said nucleic acid formulation comprises liposomes.

30. The composition of claim 28, wherein said nucleic acid formulation comprises a lipoplex.

31. The composition of any one of claims 28-30, wherein the nucleic acid is encapsulated within the lipid nanoparticles, liposomes, or lipoplex.

32. The composition of any one of claims 27-31, wherein the nucleic acid is mRNA.

33. The composition of any one of claims 27-32, wherein the methylene blue has a purity of at least 70%, 80%, 90%, 95%, or 99%.

34. The composition of any one of claims 27-33, wherein the methylene blue contains fewer than 100 ppm of elemental metals.

35. The composition of any one of claims 27-34, wherein the composition is formulated in an aqueous solution.

36. The composition of claim 35, wherein the aqueous solution comprises lipid nanoparticles and wherein the nucleic acid is encapsulated in the lipid nanoparticles.

37. The composition of claim 35 or 36, wherein the aqueous solution has a pH of or about 5 to 8, including pH of about 5, 5.5, 6, 6.5, 7, 7.5, or 8.

38. The composition of any one of claims 35-37, wherein the aqueous solution does not comprise NaCl.

39. The composition of any one of claims 35-37, wherein the aqueous solution comprises NaCl in a concentration of or about 150 mM.

40. The composition of any one of claims 35-39, wherein the aqueous solution comprises a phosphate buffer, a tris buffer, an acetate buffer, a histidine buffer, or a citrate buffer.

41. The composition of any one of claims 35-40, wherein the methylene blue is present at a concentration between about 0.1 mM and about 3 mM.

42. The composition of any one of claims 35-41, wherein the methylene blue is present at a concentration of or about 2 mM.

43. The composition of any one of claims 35-41, wherein the methylene blue is present at a concentration of or about 1 mM.

44. The composition of any one of claims 35-41, wherein the methylene blue is present at a concentration of or about 0.5 mM.

45. The composition of any one of claims 27-34, wherein the nucleic acid is a lyophilized product.

46. The composition of claim 45, wherein the lyophilized product comprises lipid nanoparticles wherein the nucleic acid is encapsulated in the lipid nanoparticles.

47. Use of the composition according to any preceding claim for the treatment of a disease in a subject.

48. The use according to claim 47, wherein the disease is caused by an infectious agent.

49. The use according to claim 48, wherein the disease is caused by or associated with a virus.

50. The use according to claim 47, wherein the disease is caused by or associated with a malignant cell.

51. The use according to claim 50, wherein the disease is cancer.

52. The composition of any one of claims 1-46 or the use of any one of claims 47-51, wherein microbial growth in the composition is inhibited by the compound.

53. The composition of any one of claim 1-46 or 52 or the use of any one of claim 47-51 or 52, wherein the composition does not comprise phenol, m-cresol, or benzyl alcohol.

54. A method of formulating a nucleic acid comprising:
adding to a composition comprising a nucleic acid and a lipid, a compound of Formula I: 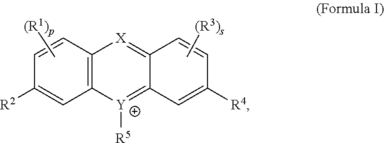
or an acceptable salt, tautomer, reduced form, or oxidized form thereof, wherein:
Y is N, S, or O;
X is N—R⁵, S, O, or C—R^{C};
R² and R⁴ are each independently —N(R^{N})₂;
each R⁵ is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or is absent;
each instance of R¹ and R³ is independently halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, optionally substituted sulfinyl, optionally substituted sulfonyl, —OR^{O}, —N(R^{N})₂, or —SR^{S};
p is 0, 1, 2, or 3;
s is 0, 1, 2, or 3;
each instance of R^{O} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or an oxygen protecting group;
each instance of R^{N} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or a nitrogen protecting group; or two R^{N} bonded to the name nitrogen atom are taken together with the intervening atoms to form optionally substituted heterocyclyl or optionally substituted heteroaryl;
each instance of R^{S} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or a nitrogen protecting group; and
R^{C} is hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, optionally substituted sulfinyl, optionally substituted sulfonyl, —OR^{O}, —N(R^{N})₂, or —SR^{S},
to prepare a formulated composition comprising the nucleic acid and the lipid.

55. The method of claim 54, wherein the formulated composition comprises lipid nanoparticles.

56. The method of claim 54 or 55, wherein the formulated composition further comprises liposomes.

57. The method of any one of claims 54-56, wherein the formulated composition further comprises a lipoplex.

58. The method of any one of claims 54-57, wherein the nucleic acid is encapsulated in the lipid nanoparticles, liposomes, or lipoplex.

59. The method of any one of claims 54-58, further comprising subsequently removing the compound of Formula I from the formulated composition.

60. The method of any one of claims 54-59, wherein the compound is a compound of Formula II: 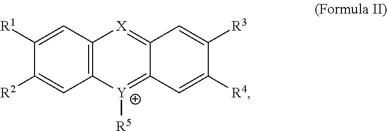
or an acceptable salt, tautomer, reduced form, or oxidized form thereof.

61. The method of any one of claims 54-60, wherein the compound is a compound of Formula III: 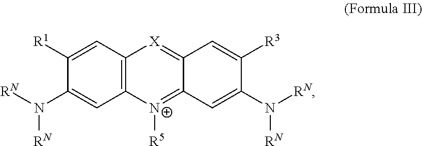
or an acceptable salt, tautomer, reduced form, or oxidized form thereof.

62. The method of any one of claims 54-61, wherein the compound is not thionine or a salt thereof.

63. The method of any one of claims 54-62, wherein the compound is methylene blue, acriflavine, toluidine blue O, safranin O, phenosafranin, leucomethylene blue or any mixture thereof.

64. The method of any one of claims 54-63, wherein the compound is methylene blue, acriflavine, safranin O, phenosafranin, leucomethylene blue or any mixture thereof.

65. The method of any one of claims 54-63, wherein the compound is methylene blue.

66. The method of any one of claims 54-65, wherein the compound has a purity of at least 70%, 80%, 90%, 95%, or 99%.

67. The method of any one of claims 54-66, wherein the compound contains fewer than 100 ppm of elemental metals.

68. The method of any one of claims 54-67, wherein the composition is formulated in an aqueous solution.

69. The method of claim 68, wherein the aqueous solution comprises lipid nanoparticles and wherein a nucleic acid is encapsulated in the lipid nanoparticles.

70. The method of claim 68 or 69, wherein the aqueous solution has a pH of or about 5 to 8, including pH of about 5, 5.5, 6, 6.5, 7, 7.5, or 8.

71. The method of any one of claims 68-70, wherein the aqueous solution does not comprise NaCl.

72. The method of any one of claims 68-70, wherein the aqueous solution comprises NaCl in a concentration of or about 150 mM.

73. The method of any one of claims 68-72, wherein the aqueous solution comprises a phosphate buffer, a tris buffer, an acetate buffer, a histidine buffer, or a citrate buffer.

74. The method of any one of claims 68-73, wherein the compound is present at a concentration between about 0.1 mM and about 3 mM.

75. The method of any one of claims 68-73, wherein the compound is present at a concentration of or about 2 mM.

76. The method of any one of claims 68-73, wherein the compound is present at a concentration of or about 1 mM.

77. The method of any one of claims 68-73, wherein the compound is present at a concentration of or about 0.5 mM.

78. The method of any one of claims 54-93, wherein the composition is a lyophilized product.

79. The method of claim 78, wherein the lyophilized product comprises lipid nanoparticles.

80. The method of claim 79, wherein the lipid nanoparticles encapsulate a nucleic acid.

81. A pharmaceutically acceptable method of processing an mRNA-lipid nanoparticle for therapeutic injection, comprising adding a reactive compound to a lipid nanoparticle, and subsequently adding an mRNA to the lipid nanoparticle-reactive compound mixture, wherein the reactive compound sequesters degradative species of the lipid nanoparticle.

82. A pharmaceutically acceptable method of conferring anti-microbial properties to an mRNA-lipid nanoparticle composition, comprising adding a reactive compound to the mRNA-lipid nanoparticle composition.

83. A pharmaceutically acceptable method of processing an mRNA-lipid nanoparticle for therapeutic injection, comprising adding an mRNA to a lipid nanoparticle, and subsequently adding a reactive compound to the lipid nanoparticle-mRNA mixture, wherein the reactive compound sequesters degradative species of the lipid nanoparticle.

84. A pharmaceutically acceptable method of processing an mRNA-lipid nanoparticle for therapeutic injection, comprising combining an mRNA, a lipid nanoparticle, and a reactive compound, wherein the reactive compound sequesters degradative species of the lipid nanoparticle.

85. A composition comprising:
a lipid nanoparticle encapsulating a mRNA, wherein the composition comprises a mRNA purity level of greater than 50% main peak mRNA purity after at least thirty days of storage.

86. The composition of claim 85, wherein the composition comprises a mRNA purity level of greater than 60% main peak mRNA purity after at least thirty days of storage.

87. The composition of claim 85 or 86, wherein the composition comprises a mRNA purity level of greater than 70% main peak mRNA purity after at least thirty days of storage.

88. The composition of any one of claims 85-87, wherein the composition comprises a mRNA purity level of greater than 80% main peak mRNA purity after at least thirty days of storage.

89. The composition of any one of claims 85-88, wherein the composition comprises a mRNA purity level of greater than 90% main peak mRNA purity after at least thirty days of storage.

90. The composition of claim 85, wherein the composition comprises a mRNA purity level of greater than 50% main peak mRNA purity after at least six months of storage.

91. The composition of any one of claims 85-90, wherein the storage is at room temperature.

92. The composition of any one of claims 85-90, wherein the storage is at greater than room temperature.

93. The composition of any one of claims 85-90, wherein the storage is at 4° C.

94. The composition of any one of claims 85-93, wherein the composition comprises a phenothiazinium dye.

95. The composition of claim 94, wherein the phenothiazinium dye is not thionine or a salt thereof.

96. The composition of claim 94 or 95, wherein the phenothiazinium dye is methylene blue.

97. A composition comprising:
a lipid nanoparticle encapsulating a mRNA, wherein the mRNA comprises intact mRNA and at least one RNA fragment, wherein the composition comprises less than 50% RNA fragments after at least thirty days of storage.

98. The composition of claim 97, wherein the composition comprises less than 60% RNA fragments after at least thirty days of storage.

99. The composition of claim 97 or 98, wherein the composition comprises less than 70% RNA fragments after at least thirty days of storage.

100. The composition of any one of claims 97-99, wherein the composition comprises less than 80% RNA fragments after at least thirty days of storage.

101. The composition of any one of claims 97-100, wherein the composition comprises less than 90% RNA fragments after at least thirty days of storage.

102. The composition of any one of claims 97-101, wherein the composition comprises less than 95% RNA fragments after at least thirty days of storage.

103. The composition of any one of claims 97-102, wherein the composition is stored for at least six months.

104. The composition of any one of claims 97-103, wherein the storage is at room temperature.

105. The composition of any one of claims 97-103, wherein the storage is at greater than room temperature.

106. The composition of any one of claims 97-103, wherein the storage is at 4° C.

107. The composition of any one of claims 97-106, wherein the composition comprises a phenothiazinium dye.

108. The composition of claim 107, wherein the phenothiazinium dye is not thionine or a salt thereof.

109. The composition of claim 107 or 108, wherein the phenothiazinium dye is methylene blue.

110. The composition of any one of claims 97-109, wherein the lipid nanoparticle comprises a ratio of 20-60% amino lipids, 5-30% phospholipid, 10-55% structural lipid, and 0.5-15% PEG-modified lipid.

111. The composition of any one of claims 97-110, wherein the lipid nanoparticle comprises a ratio of 20-60% amino lipids, 5-25% phospholipid, 25-55% structural lipid, and 0.5-15% PEG-modified lipid.

112. A method for producing a protein in a subject, comprising
administering a composition of any one of claim 1-46, 52-53, or 85-111 to a subject, wherein the nucleic acid is an mRNA and wherein the mRNA encodes for the production of a protein in the subject.

113. A syringe or cartridge, comprising a composition of any one of claim 1-46, 52-53, or 85-111.

114. An infusion pump, comprising a composition of any one of claim 1-46, 52-53, or 85-111.

115. A syringe or cartridge, comprising multiple doses of a composition of any one of claims 1-46, 52-53, 85-111.
